(19) United States
(12) Patent Application Publication
(10) Pub. No.: US 2016/0009769 A1
(43) Pub. Date: Jan. 14, 2016

(54) PROCESSES FOR ISOLATING SALICYCLIC ACID RECEPTORS

(71) Applicant: NEOPRO LABS, LLC, Bellevue, WA (US)

(72) Inventor: Hanna Skubatch, Seattle, WA (US)

(21) Appl. No.: 14/771,292

(22) PCT Filed: Mar. 3, 2014
(86) PCT No.: PCT/US14/20016

§ 371 Date: Aug. 28, 2015

Related U.S. Application Data
(60) Provisional application No. 61/771,712, filed on Mar. 1, 2013.

(51) Int. Cl. C07K 14/415; A61K 47/48; G01N 33/74; C07K 16/28

(52) U.S. Cl. CPC C07K 14/415; C07K 16/28; A61K 47/48276; G01N 33/74; C07K 2317/34; G01N 2500/10

(57) ABSTRACT

The invention provides processes for isolating salicylic acid receptor proteins (SARP) from a diverse origin using organic extraction solutions. The processes comprise the extraction of SARP from the tissue using an oarganic extraction solution, and percipitate the proteins in water, and then purified them to homoginity using reversed phase HPLC. The novel uses of SARP are in control of cellular energy and temperature homehostasis through conformational changes, cellular timekeeping mechanism, gel oscillation, and nanotechnology.

### CROSS-REFERENCE

This application claims the benefit of U.S. Provisional Application No. 61/771,712, filed Mar. 1, 2013, which application is incorporated herein by reference.

### BACKGROUND OF THE INVENTION

1. Field of the Invention

The present invention relates generally to isolated and purified proteins. These proteins relate generally to a class of salicylic acid (SA) receptor proteins (SARPs) that bind SA, aspirin (ASA) and 2,6-dihydroxybenzoic acid (2,6-DHBA). These SARPs belong to the NAD(P)H-reductase family. They behave as metabolic switch proteins that adopt different conformational states for different functions. They control the temperature set point of plants and animals alike. The conformational states are associated with at least five self-assembling pathways. When SARP bind one of the ligands, a conformational shift occurs. In particular, the invention relates to an isolated SARP from thermogenic plants and non-thermogenic plants, and from human neuroblastoma cell line SK-N-SH and from mouse brain tissue, and to related compositions and methods for: thermoregulation, treating brain diseases and disorders, studying new targets for aspirin, identifying cancer prevention and treatment, drug-screening, protecting plants against pathogens, therapeutic agent delivery, and nanotechnology.

2. Description of the Related Art

Given the widespread effects of salicylates, clearly it would be useful to delineate salicylate biological signaling pathways at a molecular level, as each component of these pathways could serve as a target for the design of drugs for humans or for crop protection products that mediate SA signaling pathways.

Unfortunately, despite the long history of use of SA and salicylate derivatives as therapeutics, the molecular mechanisms by which the salicylates mediate biological effects have not been completely defined. Although it is generally accepted that the analgesic and anti-inflammatory effects of salicylates and ASA primarily arise from inhibition of COX-2, there is growing evidence that undefined COX-independent pathways may also be involved in the anti-inflammatory actions of salicylate (Tegeder et al., 2001, FASEB J 15:2057). Even less is known about the downstream biological signaling mechanisms of salicylates as they mediate other physiological effects, such as diabetes, antitumor activity, resistance to plant pathogens, etc. The signaling components of these pathways have yet to be fully elucidated.

Many therapeutic effects of SA and ASA cannot be attributed to inhibition of COX-2 and COX-1. For example, the serum concentration of SA, but not of ASA, correlates well with anti-inflammation treatments (Cronstein et al., 1994, Inflammation 18:323; Abramson et al., 1994, Biochem Pharmacol 47:563). SA and ASA inhibit translocation of the nuclear transcriptional regulator, NF-kB into the nucleus (Cavallini et al., 2001, Biochem Pharmacol 62:141; Tegeder et al., 2001, FASEB J 15:2057). Both compounds also interfere with signaling via the MAP kinases, by inhibiting JNK and activating p38 (Schwenger et al., 1997, Proc Natl Acad Sci USA 94:2869), and by inhibiting ERK (Pillinger et al., 1998, Proc Natl Acad Sci USA 95:14540). Although ASA is a more potent inhibitor of COX-2 than SA, both compounds induce apoptosis at the same rate in B-cell lymphocytic leukemia (Bellosillo et al., 1998, Blood 92:1406). SA also induces adenosine production (Cronstein et al., 1993, Proc Natl Acad Sci USA 96:6377).

The mechanism by which SA exerts an anti-diabetic effect is unclear. Initially, the SA effect was thought to arise by direct inhibition of IKKβ, the dominant kinase in the cytokine-mediated activation of the NFkB pathway (Yuan et al., 2001, Science 293:1673), but more recent studies have implicated an IKKβ-independent mechanism as well (Jiang et al., 2003, J Biol Chem 278:180).

The signaling pathways of SA in plants are more elusive than in mammals, since COX-1 and COX-2 have not been detected in plants. SA in plants binds and inhibits several enzymes e.g., catalase (Dumer and Klessig, 1996, J Biol Chem 271:28492) and carbonic anhydrase (Slaymeker et al., 2002, Proc Natl Acad Sci 99:11640). SA also induces phosphorylation, mitochondrial alternative respiration, and inhibition of mitochondrial electron transport and oxidative phosphorylation in plants (Xie et al., 1999, Plant Physiol 120:217).

Clearly, SA and ASA in humans and plants mediate diverse biological effects not only via COX-dependent signaling pathway but also via independent pathways. Thus, there is a need in the art for identification of SARP proteins (SARPs) and other molecular components of SA-signaling pathway(s). The present invention fulfills these needs by providing a SARP that binds salicylate-related compounds, and it also offers other related advantages.

### SUMMARY OF THE INVENTION

The present disclosure contemplates an isolated and purified salicylic acid receptor protein (SARP) that binds salicylic acid at a concentration less than 10⁻⁹ molar (M). In some instances, the SARP binds salicylic acid at a concentration less than 10⁻¹⁰, 10⁻¹¹ or 10⁻¹² molar (M).

The present disclosure also contemplates an isolated and purified SARP that binds salicylic acid wherein said SARP has an in vitro volume phase transition. Such SARP can have a volume phase transition occurs at least once every 30 minutes, 20 minutes, 15 minutes, 10 minutes, 9 minutes, 8 minutes, 7 minutes, 6 minutes, 5 minutes, 4 minutes, 3 minutes, 2 minutes, or 1 minute.

An isolated and purified SARP of the disclosure, according to DLS, has a uniform size of ˜800 nm and ˜300 nm alternating at repeating intervals. Such isolated and purified SARP can have at least two conformational states or two conformational states.

In some instances, an isolated and purified SARP has an ESI-MS spectra with maxima at one or more of the following: m/z 854, 900, 924, 1178, 1220 or other maxima as shown in the figures herein.

The disclosure herein also contemplates an antibody or antibody fragment that selectively binds an SARP herein, or a single conformation of the SARP herein. Such antibody(ies) can be in a kit. For example, in one instance, a kit can have a first antibody to state A of the SARP and a second antibody to state B of the SARP.

The disclosure herein also contemplates a method for identifying agents that modulate SARP comprising performing a competition assay between a first agent selected from the group consisting of SA, ASA, and 2,6-DHBA and a second agent and their binding affinity to an SARP, and selecting the second agent that outcompetes binding of the first agent to the SARP for drug development.

The disclosure herein also contemplate a method for assaying for a metabolic condition in a subject comprising: assaying for the presence of a state A or state B conformation of a SARP in a sample from the subject and providing a report indicating if the subject is susceptible to or is experiencing such metabolic condition.

The disclosure herein contemplates an isolated and purified salicylic acid receptor protein (SARP) having at least 50%, 60%, 70%, 80%, 90%, 95%, 99% amino acid sequence homology to the isolated SARP described herein.

The disclosure herein also contemplates a fragment of an isolated and purified SARP wherein said fragment selectively binds SA, ASA or 2,6-DHBA. For example, such fragment can be a peptide having SEQ ID NO: 1, 2, or 102.

A novel SARP was discovered in the thermogenic inflorescence of Sauromatum guttatum and other thermogenic plants as well as Arabidopsis thaliana (Skubatz and Howald, 2013, Protein J 32:197). SARP switches between an expanded state A and a more compact state (state B) with the appearance of sub-states as well in the presence of SA, ASA, and 2,6-DHBA (Skubatz and Howald, 2013, Protein J 32:399; Skubatz et al., 2013, Intrinsically Disordered Proteins 1:e26372) SARP is also found in the human SK-N-SH cell line and in mouse brain tissue (Skubatz and Howld, 2013, Protein J 32:641). The molecular mass of human SARP is in the same range as of its plant counterpart, 34,140±34 Da. The charge state distribution (CSD) of the human SARP is identical to its plant counterpart from the Sauromatum appendix during heat-production. Human SARP was present in the compact state when it was purified from the SK-N-SH cell line When these cells were treated with SA (10 μM) a shift to a much more compact conformation was observed. It seems that the potential of SARP to respond to salicylic acid was conserved. These results may reveal the existence of a thermoregulation system that is evolutionarily conserved and is operating by conformational changes. This regulatory activity behaves as a metabolic switch that switching on and off heat-production. SARP can be a metabolic switch in the cytoplasm and mitochondria. For example, in state A (the expanded form) SARP is inactive in terms of thermogenicity but is active or involved in heat-production in state B. On the day of heat-production, the level of SA in the Sauromatum appendix is such that SARP is mostly in the compact state. However, if SA level fluctuates, SARP can be either in state B (compact) or state A (extanded) and consequently, can trigger oscillatory thermogenicity. In a more complex model, the cytoplasmic and the mitochondrial switch will have to be to be in state B. There are four possibilities for the output for both switches. When both switches are in states B, a singular event of heat-production occurs. Other options are cytoplasmic SARP in state A and mitochondrial SARP in either state A or B. Or, cytoplasmic SARP in state B and mitochondrial SARP in state A.

SA is synthesized on D-1 morning in the Sauromatum appendix (Raskin et al., 1989, Proc Natl Acad Sci USA 86: 2214). On one day prior to heat-production the level of SA starts to rise in the afternoon and reaches its maximum in the late evening. Addition of SA on D-1 morning to a section of the appendix will trigger thermogenicity and a temperature rise up to 32° C. the next morning, whereas an untreated section will stay at ambient temperature. These changes in the SA level coincide with the changes in SARP conformation. Binding of SA through unfolded or partially unfolded intermediates can provide a kinetic advantage through the “fly-casting” mechanism (Shoemaker et al., 2000, Proc Natl Acad Sci USA 97:8868). According to this mechanism protein folding is coupled with binding (Sugase et al., 2007, Nature 447:1021).

Laser dynamic light scattering (DLS) measurements of SARP in water demonstrated the unusual physical properties of SARP to condense and relax. Upon addition of dry SARP to distilled water at a final concentration of 0.1 pM, micelles were formed with a uniform size of ˜800 nm. Organized droplets are seen under the light microscope when SARP solution is spread on a glass slide. Upon addition of DTT or mercaptoethanol the micelle size reduces to ˜300 nm, and after 20 min, the size increases back to ˜800 nm. Addition of 1 pM SA, the micelle size. immediately decreased. SARP molecules become intertwined and form a gel that expands and shrinks in an oscillatory manner, every 4-5 min. The swelling and shrinking were discontinuous. Addition of 10 μM ATP stops the oscillation for ˜30 min. and then it is resumed at the same kinetics.

The biophysical properties of this protein are remarkable. It is self-assembled in aqueous solutions into micrometer sizes of organized aggregates. The assembly of this protein produced a broad range of cyclic and linear assembles that resemble micelles, rods, lamellar micelles, as well as vesicles. The assembles displayed an unusual behavior in that they were also assembling to form network structures. The molecules entangle with each other and formed branched, interconnected networks. The data lay the foundation for exploring the mechanism of folding, misfolding, and aggregation of SARP. It illustrates the potential role of SARP in storage and secretion of odoriferous volatiles during heat-production.

In certain embodiments SARP is isolated using organic solutions. Tissues are ground n a juicer grinder in an organic extraction solution containing 50% IP, 2 mM β-mercaptoethanol, 0.5 mM EDTA, 5 mM butylated hydroxy-toluene, 0.1% tocopherol acetate, 0.1% polyvinylpoly-pyrrolidone (insoluble, Sigma-Aldrich Corp.) in 40 mM imidazole/HCl buffer, pH 7.0, at a weight/volume ratio of 1 g of tissue per 5 ml of extraction solution. The extract is centrifuged at 15,000 g for 10 min and the pellet is discarded. A strong cationic resin (DOWEX X-50, Sigma-Aldrich Corp.) is added to the supernatant (30 mg resin per ml) and stirred for 2.5 h at 4° C. The mixture is then centrifuged at 30 g for 2 min, and the supernatant is discard. The Dowex resin is washed several times, first with the extraction solution and finally with distilled water until a clear wash is obtained. Subsequently, the resin is again washed 2 times with 0.15 M phosphate buffer, pH 7.0. SARP is eluted from the resin with 0.4 M phosphate buffer, pH 7.0. The protein solution is dialyzed using a 12-14 kDa cut-off dialysis bag against 3 L of distilled water at 4° C., the water is changed twice during 24 h. The content of the dialysis bag is removed and centrifuged at 10,000 g for 15 min. The pellet is used for SARP purification.

In another embodiment an isolated SARP comprising a polypeptide of ˜34.1 kDa that is isolated from a biological sample by a process comprising the steps of: (a) extracting the biological sample in a solvent to obtain a soluble extract free from insoluble material, wherein the solvent is selected from the group consisting of (i) a solvent comprising at least 50% isopropanol, (ii) a solvent comprising isopropanol: acetone: toluene at a ratio of 1:2:1 (iii) a solvent having a solvent polarity/polarizability (SPP) scale value of from ˜0.655 to 0.900. According to certain embodiments SARP is soluble in a solvent selected from toluene, aqueous isopropanol, isopropanol, and water. In a further embodiment aqueous isopropanol comprises at least 25% isopropanol by volume and in another further embodiment aqueous isopropanol comprises at least 95% isopropanol by volume.

In certain embodiments SARP is purified using a reversed-phase high-performance liquid chromatography (RP-HPLC) on-line to elecrosray ionization-mass spectromer (ESI-MS). An HPLC system consisting of a Shimadzu LD-10AD solvent delivery system and a Shimadzu SPD-10AV UV-Vis variable detector (Shimadzu Scientific Instruments, Inc.) fitted with a Rheodyne 8125 manual injector (Rheodyne Inc.) is used in the purification of SARP. The pellet is dissolved in 1% TFA and diluted 20 times with distilled water and reduced with 1 mM DTT. SARP solution is then fractionated using perfusion chromatography using a 2.1×150 mm, POROS® R1 poly(styrene-divinylbenzene) column slurry packed in-house with 20 μm particles (Applied Biosystems). The mobile phase consisted of 0.05% (v/v) aqueous TFA (solvent A) and 0.05% aqueous TFA in ACN:IP at a ratio 2:1. SARP solution is injected onto the HPLC column using a 100 μl injection loop and the protein is eluted under a linear gradient from 10 to 100% of solvent B at a flow rate of 1 ml min⁻¹ and is monitored by absorbance at 214 nm and MS signals ((TIC and MaxEnt). SARP fractions are combined and dried.

It is an aspect of the present invention to provide a method of detection and characterization of SARP using RP-HPLC/ESI-MS. This is accomplished using on-line splitting of 5 to 10% of the effluent from the UV detector to a Micromass Quattro II tandem quadrupole MS fitted with an ESI source (Waters Inc.) via a variable splitter allowing manual fraction collection. This instrument is operated in a positive ESI mode at a probe tip voltage of 3.6 kV and a cone voltage of 45 V. The source and nebulizer temperatures are maintained at 150 and 400° C., respectively, using N₂ as both nebulizing and bath gas. The instrument is calibrated using water clusters over the range of 100-2400 Da with data acquisition is carried out from m/z 500 to 2000 in the continuum-scanning mode at 450 Da sec¹. Instrument tuning, mass calibration, data acquisition, processing and display are accomplished using MassLynx™ and MaxEnt™ software (Waters Inc.). This procedure provided a protein molecular mass with an accuracy of ˜0.1%.

It is an aspect of the present invention to provide an isolated SARP that binds SA, ASA, and 2,6-DHBA comprising a polypeptide of ˜34.1 kDa, wherein the protein comprises at least one binding site for a ligand, the ligand consisting of (a) a salicylate-related compound, (b) and a nucleotide triphosphate that is selected from a nucleotide triphosphate such as ATP or guanosine triphosphate (GTP), (c) and a nicotinamide adenine dinucleotide such as NAD, NADH, NADP, and NADPH.

In certain embodiments the presence of a shift in CSD detected using ESI-MS is used for the detection and characterization of SARP. In other embodiments in the presence of a salicylate-related compound the CSD of SARP is altered. In another embodiment SARP CSD is altered under conditions selected from (i) changes in NAD(P)/NAD(P)H and (ii) ATP/ADP and GTP/GDP ratio.

In certain embodiments in the absence of a ligand an ESI-MS spectrum of SARP has at least one peak of a protein ion at m/z 1178 and a second peak at m/z 924, and in the presence of a salicylate-related compound a change in the CSD of SARP comprises at least one change in the magnidute of a protein ion; in certain further embodiments the change occurs in the presence of a salicylate-related compound and, (i) changes in NAD(P)/NAD(P)H and (ii) ATP/ADP and GTP/GDP ratio.

In another embodiment SARP is capable of transferring two electrons to an electron acceptor, which in certain still further embodiments is selected from the group consisting of MTT, NBT and Neo-NBT, and cytochrome c.

In other embodiments SARP is isolated from a thermogenic plant that belongs to a family selected from the group consisting of Araceae, Nymphaeaceae and Cycadaceae. In one embodiment the plant is Sauromatum guttatum. In other embodiments the plant is selected from the group consisting of Arum itallcum, Amorphophallus konjac and Dracunculus vulgaris. In another embodiment the plant is Victoria cruziana. In another embodiment the plant is Encepalartos feros. In other embodiments SARP is isolated from an Arabidopsis plant. In other embodiments SARP is isolated from neuronal cell or tissue, which in certain further embodiments is a human cell line or a mouse brain tissue.

In certain embodiments the sizes of SARP oligomers are determined using dynamic light scattering (DLS). In other embodiments in the presence of a salicylate-related compound SARP oscillates in a constant rate between condesed and relaxed structures.

In another embodiment SARP is capable of self-assembly using at least five different assembly pathways, which in certain further embodiments comprises a linear structure when self-assembly, and wherein more complex structures comprise, for example, a dendrimer structure when self-assembly occurs. In certain further embodiments the structure comprises vesicle structures.

Turning to another aspect that provides a method of identifying an agent that alters a biological effect, comprising (a) contacting an isolated SARP with a candidate agent under conditions and for a time sufficient to permit interaction between said SARP and said agent, wherein (i) SARP comprises a polypeptide of ˜34.1 kDa having an ESI-MS spectrum which comprises a known spectrum of SARP from Suromatum inflorescence, and (ii) SARP comprises at least one binding site for a ligand that is selected from the group consisting of a salicylate-related compound, and from ATP, GTP, NAD(P), NAD(P)H; and (b) comparing a level of binding of a ligand to SARP in the absence and presence of the candidate agent, wherein an altered level of binding in the presence of the agent indicates the agent alters a biological effect.

In another embodiment a method is provided of identifying an agent that alters a biological effect, comprising: (a) contacting an isolated SARP, an electron acceptor molecule and a candidate agent under conditions and for a time sufficient to permit transfer of at least one electron from SARP to the electron acceptor, wherein (i) SARP comprises a polypeptide of ˜34.1 kDa having ESI-MS spectrum with one protein ion at m/z 1178 or a protein ion at m/z 924 or a protein ion at m/z 1314 in the absence of a salicylate-related compound and which comprises at least one change in its CSD in the presence of a salicylate-related compound, and (ii) SARP comprises at least one binding site for a ligand that is selected from the group consisting of a salicylate-related compound, and from ATP, GTP, NAD(P), NAD(P)H; and (b) comparing a level of electron transfer from SARP to the electron acceptor in the absence and presence of the agent, wherein an altered level of electron transfer in the presence of the candidate agent indicates the agent alters a biological effect.

According to another embodiment a method is provided for identifying an agent that alters a biological effect, comprising: (a) contacting an isolated SARP with a candidate agent under conditions and for a time sufficient to permit interaction between said SARP and said agent, wherein (i) SARP comprises a polypeptide of ˜34.1 kDa having a known CSD detected using ESI-MS and which comprises at least at least one one protein ion at m/z 1178 or a protein ion at m/z 924 or a protein ion at m/z 1314 in the presence of a salicylate-related compound, and (ii) SARP comprises at least one binding site for a ligand that is selected from the group consisting of a salicylate related-compound, and from ATP, GTP, NAD(P), NAD(P)H; and (b) comparing, in the absence and presence of the candidate agent, a conformational change of SARP at one or a plurality of time points, wherein an altered conformational of SARP in the presence of the agent relative to the conformational state of SARP in the absence of the agent indicates the agent alters a biological effect.

According to another embodiment there is provided a method of identifying an agent that alters a biological effect, comprising: (a) contacting an isolated SARP with a candidate agent under conditions and for a time sufficient to permit interaction between said SARP and said agent, wherein (i) SARP comprises a polypeptide of ˜34.1 kDa having a known CSD detected using ESI-MS and which comprises a change in the CSD in the presence of a salicylate-related compound, and (ii) SARP comprises at least one binding site for a ligand that is selected from the group consisting of a salicylate related-compound, and from ATP, GTP, NAD(P), NAD(P)H, and (b) comparing, in the absence and presence of the candidate agent, an oligomeric state of SARP at one or a plurality of time points, wherein an altered oligomeric state of SARP in the presence of the agent relative to the oligomeric state of the protein in the absence of the agent indicates the agent alters a salicylate biological effect.

In certain further embodiments the biological effect is selected from the group consisting of: treatment of pain, plant pathogen resistance, malignant cell growth, decreased interaction with extracellular matrix and cytoskeleton, an altered NAD(P), NAD(P)H or ATP, GTP levels in a cell, changes in core body temperature, inflammation, and an undesired side-effect of a salicylate-related compound. In other further embodiments SARP is isolated from a thermogenic plant, or from a mammal, which in certain further embodiments is selected from a primate, a leporida (rabbit), a caviida (guinea pig), a rodent, a bovida and a suida (pig). In certain further embodiments the primate is selected from a human and a non-human primate, and in other further embodiments the rodent is selected from a mouse, a rat, a hamster and a guinea pig.

The invention also provides in certain embodiments an antibody that specifically binds SARP as described here. In another embodiment a protein complex for biological delivery of an agent is provided, comprising a plurality of isolated SARP molecules, wherein: (i) SARP comprises a polypeptide of ˜34.1 kDa having having a known CSD detected using ESI-MS in the absence of a salicylate-related compound and which comprises at least one one protein ion at m/z 1178 or protein ion at m/z 924 in the presence of a salicylate-related compound, (ii) SARP comprises at least one binding site for a ligand that is selected from the group consisting of a salicylate-related compound and from ATP, GTP, NAD(P), NAD(P)H, and (iii) the complex is present in an oligomeric state that is selected from the group consisting of gels and aggregates.

In another embodiment provided for SARP complex for biological delivery of an agent, comprising a gel which comprises a plurality of isolated SARP molecules, wherein: (i) SARP comprises a polypeptide of ˜34.1 kDa having a known CSD detected using ESI-MS in the absence of a salicylate-related compound and which comprises at least one protein ion at m/z 1178 or protein ion at m/z 924 in the presence of a salicylate-related compound, and (ii) SARP comprises at least one binding site for a ligand that is selected from the group consisting of a salicylate related-compound and from ATP, GTP, NAD(P), NAD(P)H. In certain further embodiments the complex comprises an oscillating gel.

In yet another embodiment a method is providedfor identifying an agent that alters a biological effect, comprising (a) contacting an isolated SARP with a candidate agent under conditions and for a time sufficient to permit interaction between said SARP and said agent, wherein (i) SARP comprises a plant reductase polypeptide selected from plant polypeptides comprising amino acid sequences set forth in the group consisting of SEQ ID NOS:2-101, said plant reductase polypeptide having a CSD comprises at least one protein ion at m/z 1178 or protein ion at m/z 924 in the absence of a salicylate-related compound and which comprises at least one change in the magnidute of the CSD in the presence of a salicylate-related compound, and (ii) SARP comprises at least one binding site for a ligand that is selected from the group consisting of a salicylate-related compound and a from ATP, GTP, NAD(P), NAD(P)H; and (b) comparing a level of binding of a ligand to SARP in the absence and presence of the agent, wherein an altered level of binding in the presence of the agent indicates the agent alters a biological effect.

In another embodiment a method is provided for identifying an agent that alters a biological effect, comprising: (a) contacting an isolated SARP, an electron acceptor molecule and a candidate agent under conditions and for a time sufficient to permit transfer of at least one electron from SARP protein to the electron acceptor, wherein (i) SARP comprises a plant reductase polypeptide selected from plant polypeptides comprising amino acid sequences set forth in the group consisting of SEQ ID NOS:3-10, said plant reductase polypeptide having a CSD comprises at least one one protein ion at m/z 1178 or protein ion at m/z 924 in the absence of a salicylate-related compound and which comprises at least one change in the magnidute of the CSD in the presence of a salicylate-related compound, and (ii) SARP comprises at least one binding site for a ligand that is selected from the group consisting of salicylate related-compound and from ATP, GTP, NAD(P), NAD(P)H; and (b) comparing a level of electron transfer from SARP to the electron acceptor in the absence of the candidate agent to the level of electron transfer in the presence of the agent, wherein an altered level of electron transfer in the presence of the agent indicates the agent alters a biological effect.

Another embodiment provides a method for identifying an agent that alters a biological effect, comprising: (a) contacting an isolated SARP with a candidate agent under conditions and for a time sufficient to permit interaction between said SARP and said agent, wherein (i) SARP comprises a plant reductase polypeptide selected from plant polypeptides comprising amino acid sequences set forth in the group consisting of SEQ ID NOS:3-101, said plant reductase polypeptide having a CSD comprises at least one one protein ion at m/z 1178 or protein ion at m/z 924 in the absence of a salicylate-related compound and which comprises at least one change in the magnidute of the CSD in the presence of a salicylate-related compound, and (ii) SARP comprises at least one binding site for a ligand that is selected from the group consisting of a salicylate related-compound and a nucleotide triphosphate which is selected from ATP, GTP, NAD(P), NAD(P)H; and (b) comparing, in the absence and presence of the candidate agent, an oligomeric state of SARP at one or a plurality of time points, wherein an altered oligomeric state of SARP in the presence of the agent relative to the oligomeric state of SARP in the absence of the agent indicates the agent alters a biological effect.

In certain further embodiments the biological effect is selected from the group consisting of: plant pathogen resistance, temperature change, and redox sensing. In another embodiment the invention provides a method of detecting an altered level of resistance to a pathogen in a plant, comprising detecting at two or more time points a level or conformation of SARP at a location in a biological sample comprising a cell derived from the plant, wherein said location is selected from a cell-associated location and a non-cell-associated location, and wherein an increase in the level or conformation of SARP at the location at a later time point relative to the level or conformation of SARP at an earlier time point indicates an altered resistance level. In another embodiment the invention provides a method of altering a salicylate biological effect in a plant, comprising contacting the plant with a protein selected from those described above and a plant reductase polypeptide that is selected from plant polypeptides comprising amino acid sequences set forth in the group consisting of SEQ ID NOS:3-101, wherein the salicylate biological effect comprises resistance to frost damage. In another embodiment the invention provides a method of altering a salicylate biological effect in a plant, comprising sensing changes in NAD(P)/NAD(P)H ratio.

In certain further embodiments the biological effect is selected from the group consisting of: treatment of pain, changes in core body temperature, and redox sensing. In another embodiment the invention provides a method of detecting an altered level of pain in a human, comprising detecting at two or more time points a level or conformation of SARP at a location in a biological sample comprising a cell line derived from human, wherein said location is selected from a cell-associated location and a non-cell-associated location, and wherein an increase in the level or conformation of SARP at the location at a later time point relative to the level or conformation of SARP at an earlier time point indicates an altered resistance level. In another embodiment the invention provides a method of altering a salicylate biological effect in a human, comprising contacting the human with a protein selected from those described above and a plant reductase polypeptide that is selected from plant polypeptides comprising amino acid sequences set forth in the group consisting of SEQ ID NOS:3-101, wherein the salicylate biological effect comprises reduction in body temperature. In another embodiment the invention provides a method of altering a salicylate biological effect in a human, comprising sensing changes in NAD(P)/NAD(P)H ratio.

These and other aspects of the present invention will become apparent upon reference to the following detailed description and attached drawings. All references disclosed herein are hereby incorporated by reference in their entireties as if each is incorporated individually.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1. Purification of SARP from thermogenic Sauromatum appendix using perfusion chromatography, RP-HPLC-ESI-MS. TIC (total ion current) chromatogram with solvent gradient overlay (A); absorbance at 214 nm (B); ion current chromatogram of m/z 1178, corresponding to [M+29H]²⁹⁻ CSD of the protein (C). Solvent A is 0.05% TFA in aqueous solution and solvent B is 0.05% aqueous TFA in ACN:IP at a ratio 2:1.

FIG. 2. ESI-MS of SARP from thermogenic Sauromatum appendix. Mass spectrum at the left displays the charge state envelope of the protein (A), and the assigned positive charge states (top) are indicated above the peaks of SARP ion masses. The corresponding deconvaluted mass spectrum of a single protein with the molecular mass of 34,146±34 Da (B)

FIG. 3. ESI mass spectra of SARP from Sauromatum appendix at different stages of development. SARP was extracted from 15 g of D-4 appendix; 20 g of D-3; 20 g of D-2 appendix; 105 g of D-1 appendix. The mass determination corresponding with the envelopes was consistent with a protein of a molecular weight of 34,146±34 Da. The background ions arose from impurities from the plant extract were plotted in gray lines and the envelope in black lines. D-4 is deemed “state A” and D-Day is “state B” (FIG. 2)

FIG. 4. ESI mass spectra of mitochondrial and cytoplasmic SARP from a D-day appendix. SARP was extracted from 85 g of D-day appendix (A), and 1 mg of Percoll-purified mitochondria from D-day appendix (B). SARP was also extracted from 19 g of D-1 appendix was incubated for 2 h in 6 M guanidine-HCl and purified on the RP-HPLC system (C). SARP envelope was skewed because of impurities from the plant extract and guanidine-HCl. For Percoll-purified mitochondria the data show one major envelope with a molecular mass of 34,140±18 Da.

FIG. 5. ESI mass spectra of Arabidopsis leaves and Encephalartos feros male cones. SARP was extracted from 200 g of 4 week-old Arabidopsis green leaves and 40 g of Encephalartos feros male cones during the week of thermogenicity. Deconvolution of the data using MaxEnt showed a prominent mass of 34,130 Da for SARP from Arabidopsis (A) and 34,150 for SARP from E. Feros (C). These molecular mass are well within the molecular range of SARP from the Sauromatum appendix, 34,140±34 Da.

FIG. 6. In vitro formation of spherical ensembles by the purified SARP from the Sauromatum appendix. Representative micrographs of various morphologies were organized in a conceptual model of an assembly pathway. Bar: 12.5 μm.

FIG. 7. In vitro formation of vesicles morpholgy by the purified SARP from the Sauromatum appendix. Representative micrographs of various morphologies were organized in a conceptual model of a second assembly pathway. Bar: 12.5 μm.

FIG. 8. In vitro formation of rod morphologies by the purified SARP from the Sauromatum appendix. Representative micrographs of various morphologies were organized in a conceptual model of a third assembly pathway. Bar: 12.5 μm.

FIG. 9. In vitro formation of film morphologies by the purified SARP from the Sauromatum appendix. Representative micrographs of various morphologies were organized in a organized in a conceptual model of a fourth assembly pathway. Bar: 12.5 μm.

FIG. 10. In vitro formation of dendrimer morphologies by the purified SARP from the Sauromatum appendix. Representative micrographs of the various ensembles were assembled in order to get a conceptual model of a fifth assembly pathway. Bar: 12.5 μm.

FIG. 11. SARP was purified from D-day appendices using RP-HPLC-ESI-MS. Purified SARP was solubilized in distilled water and analyzed using DLS. The volume values of SARP alternated between 800 and 300 nm diameter every 4-5 min with a steep jump. T, period.

FIG. 12. ESI mass spectra of SARP from Sauromatum appendix after treatment with SA and ASA at different concentrations.

FIG. 13. ESI mass spectra of SARP from Sauromatum appendix after treatment with 2,6-DHBA. Sections of D-2 and D-3 appendices were incubated overnight in 100 μM solutions of 2,6-DHBA and the next day SARP was extracted. The ESI-MS spectra is different that that of ASA and SA. It is more expanded, lower m/z values.

FIG. 14. UV spectra of SAR in the presence of 2,6-DHBA. Freshly dissolved SAR was incubated with 2,6-DHBA and samples were taken for spectral analysis at the same time when the effect of SA on SAR was determined. The data present acquired spectra after baseline subtraction of each compound added to SAR. Every 3 min. after the addition of SAR a spectrum was recorded. A, The first 6 spectra were downward relative to the baseline (solid line). The following spectra were upward (broken line). (ME, β-mercaptoethanol). Incubation of NADH and SA with 1 pM SARP resulted in a shift in a band from 197 to 230 and another peak was observed at 260 nm. When azide was added a broad band was formed from 190-230 nm suggesting that SARP molecules exhibited a wide range of oxidation states. Other phenolic compounds such as 2,6-DHBA and 3,4-DHBA did not exert the same effect on SARP.

FIG. 15. UV spectra of SAR in the presence of GTP, SA, and ATP. Freshly dissolved SARP was incubated with GTP and samples were taken for spectral analysis. Later, ME and SA were added to the sample, and finally ATP was added to the sample. The data present acquired spectra after baseline subtraction of each compound added to SARP. Every 3 min. after the addition of SARP a spectrum was recorded. (ME, β-mercaptoethanol). Incubation of SA and GTP and ATP with 1 pM SARP resulted in the appearance of new bands from 197 to 230 and another peak was observed at 280 nm.

FIG. 16. Purification of SARP from SK-N-SH cells using perfusion chromatography. SARP was purified using RP-HPLC/ESI-MS. TIC chromatogram with solvent gradient overlay (A). Ion current chromatograms of m/z 1139 (B) and 1178, (C) corresponding to [M+30H]³⁰⁺ and [M+29H]²⁹⁻ charge states of the protein. Solvent A is 0.05% TFA in aqueous solution and solvent B is 0.05% aqueous TFA in ACN:IP at a ratio 2:1.

FIG. 17. Electrospray ionization mass spectrua of SARP from the SK-N-SH cells and Sauromatum appendix. Mass spectrum at the left displays the CSD of SARP from SK-N-SH cells (A), and the corresponding deconvoluted mass spectrum of a single protein with the molecular mass of 34,140±34 Da (B). The presence of several protein ions carrying similar number of charges is evident in the spectrum. The mass spectrum of SARP from the Sauromatum appendix during heat-production (C), and its corresponding deconvoluted mass spectrum (D). The assigned positive charge states (top) are indicated above the peaks of SARP ion masses.

FIG. 18. Transformed ESI mass spectrum of SARP purified from mouse brain. The protein extracted from an entire brain (˜0.5 g) from a 5 month-old mouse was analyzed using RP-HPLC/ESI-MS. ESI mass spectrum revealed at least six charged ions of SARP. Deconvolution of the data using MaxEnt showed one major protein with a prominent mass of 34,120 Da and a second one with a mass of 34,140 Da.

### DETAILED DESCRIPTION OF THE INVENTION

The present invention relates to the discovery of a novel class of SARP. SARPs possess non-linear, dynamic physicochemical characteristics upon exposure to SA, ASA, and/or 2,6-DHBA. These proteins possess highly unexpected properties, including solubility in non-polar, organic solvents such as isopropanol. Depending on the chemical environment, SARP has at least two global conformational states that that can switch from one state to the other and several sub-states. SARP exhibits mechanical oscillation at picomolar concentrations. Without wishing to be bound by theory, the nonlinearity is characteristic of charge transfer between the well-defined assembled structures formed by SARP under certain conditions, such as in the presence of SA or a salicylate-related compound. These structures may be either branched from a central core, or may instead form a membrane-like structure. Each conformation may have several energetic species, and they may be involved in the mechanical periodic oscillation.

The isolated SARP of the present invention contain at least one binding site for a ligand, which ligand may be a salicylate-related compound and/or ATP or GTP, NAD(P), NAD(P)H. Surprisingly, SARP is capable of self-assembly into a variety of different oligomeric structures depending on the solution conditions. SARP of the present invention further has the unexpected capability of forming a gel in aqueous conditions, the gel being capable of periodic oscillations that result in alternating condensed and relaxed oligomeric states. In view of the unusual properties of SARP described herein, and given the multiple biologic roles ascribed to salicylate-related compounds, the present invention provides useful compositions and methods for drug screening, for identifying agents to protect plants against pathogens, for therapeutic agent and drug delivery and for nanotechnology, as well as other related advantages.

SARPs described herein may be used in high-throughput screening for agents that interact with these proteins to identify new drugs and pesticides. Such screening, using one or more defined molecular targets (e.g., SARPs or components of a SARP signaling pathway), will be considerably less time-consuming, less labor-intensive, and less expensive than current screening assays for SA pathway-active agents that are performed using salicylate treatment of in vivo plant or animal models. Identification of components of the SA signaling pathways may therefore also be useful for the design of drugs suitable for use in any SARP pathway related disorder, such as cancer, cardiovascular and/or metabolic disease, and for production of plants capable of enhanced SAR.

### 1. Isolation of SARP

The invention thus relates in part to an isolated SARP comprising a polypeptide of ˜34.1 kDa, wherein SARP comprises at least one binding site for a ligand that may be a salicylate-related compound or ATP or GTP. The term “isolated” means that the material is removed from its original environment (e.g., the natural environment if it is naturally occurring). For example, a naturally occurring protein or polypeptide present in a living plant or animal is not isolated, but the same protein or polypeptide, separated from some or all of the co-existing materials in the natural system, is isolated. Such proteins or polypeptides could be part of a composition, and still be isolated in that such composition is not part of its natural environment. Certain embodiments relate to fragments, derivatives or variants of SARP, including chemical or enzymatic cleavage products of the isolated SARPs described herein, or chemically modified preparations of SARP, or minor structural variants which retain the physicochemical properties of SARPs described herein, such as naturally occurring variants that differ insubstantially by virtue of the subject or biological source from which they are obtained, for instance, due to expected genetic variations in outbred source subjects. Included within such embodiments are fragments of the herein described SARP. As described in greater detail below, identification of the presence of such a SARP peptide fragment that comprises at least one SARP can be accomplished readily and without undue experimentation, for instance, by assaying SARP fragments for characteristic electron donor/acceptor properties using methodologies described in the Examples, or other techniques known in the art for establishing that a composition is capable of acting as an electron donor or an electron acceptor. According to non-limiting theory, based on the extraction procedures for isolating SARP and on the assay conditions for demonstrating SARP behavior, the embodiments described here are relating to SARP fragments that comprise at least one SARP activity. SARP fragments may have a variety of uses, for instance, in photonic systems that transform information storage and transmission from the electrical to the optical regime, including high speed optical modulators, ultrafast optical switches, and high density optical data storage media.

As described in greater detail below, the isolated SARP of the invention has been characterized with respect to its molecular mass using ESI/MS, where according to this methodology the protein was determined to comprise a polypeptide of “˜” 34.1 kDa. In this context, “˜” will be understood to encompass deviations from the apparent assigned molecular mass of 34.1 kDa by no more than ten %, preferably no more than five %, more preferably no more than three %, more preferably no more than two %, more preferably no more than one %, and still more preferably by no more than one-half of one %, which deviations may, according to non-limiting theory, result from any one or more of potential technical limitations of the instrumentation, methodology and/or technique used to characterize SARP, natural variations as a function of the particular subject or biological source from which it is isolated, or from presently unrecognized unusual attributes of the protein that influence its behavior when subjected to the characterization conditions described herein.

As is well known to those skilled in the art, proteins are comprised of amino acids covalently assembled to one another via peptide linkages to form polypeptides, which typically may comprise linear polymers of the twenty common naturally occurring amino acids (alanine, cysteine, aspartic acid, glutamic acid, phenylalanine, glycine, histidine, isoleucine, lysine, leucine, methionine, asparagine, proline, glutamine, arginine, serine, threonine, valine, tryptophan, tyrosine) as well as any of the less common modified amino acids or amino acid derivatives (e.g., 4-hydroxyproIine, 5-hydroxylysine, e-N-methylysine, 3-methylhistidine, desmosine, isodesmosine, phenylglycine (Phg), 2,3-diamino butyric acid (Dab), 2,3-diamino propionic acid (Dap), p-methylaspartate (MeAsp), cyclohexylalanine (β-Cha), norieucine (Nle), norvaline (Nvl), isonipecotic acid (Ina), pipecolic acid (homoproline) (Pip or hPro), p-aminophenylacetic acid (Apa), 2-aminobutyric acid (Abu), sarcosine (Sar or N-methylglycine or MeGly), 6-amino-hexanoic acid (Ahx), 3- or 4-mercaptoproline derivatives, N⁵-acetyl-N⁵-hydroxy-L-omithine, α-N-hydroxyamino acids, etc., or other known modified amino acids).

Proteins may also comprise covalent modifications to constituent polypeptide chains, for example, posttranslational modifications such as oxidation, phosphorylation, alkylation, acylation, methylation, N-linked and/or O-linked glycosylation, C-mannosylatbn, glypiation/glycosylphosphatidylinositol modification, or phosphoglycation, all of which can be identified according to art-established criteria (e.g., Spiro, 2002 Glycobiol. 12:43R).

### 2. Extractability of SARPs in Different Solvents

As noted above, SARP of the present invention exhibit unusual hydrophobia properties, including solubility in relatively non-polar solvents. Hence, in certain preferred embodiments such relatively non-polar solvents typically have a solvent polarity/polarizability (SPP) scale value of from ˜0.655 to 0.900 using the SPP scale of Catalan et al. (e.g., 1995 Liebigs Ann. 241; see also Catalan, 2001 In: Handbook of Solvents, Wypych (ed.), Andrew W. Publ. NY, and references cited therein; [online] (retrieved on 1 Aug. 2003) http://www. uam.es/personal_pdi/ciencias/catalandepaz/web_solventsescalas/application.htm>), according to which, for example, water has a SPP value of 0.962, toluene a SPP value of 0.655, and 2-propanol (isopropanol) a SPP value of 0.848. Methods for determining the SPP value of a solvent based on UV-VIS measurements of the 2-N,N-dimethyl-7-nitrofluorene/2-fluoro-7-nitrofluorene, probe/homomorph pair, have been described (Catalan et al., 1995). Solvents with SPP values between 0.655 and 0.900 (whether as pure single-component solvents or as solvent mixtures of two, three, four or more solvents; for solvent miscibility see, e.g., Godfrey (1972 Chem. Technol. 2:359), and other solvent systems in which SARPs are soluble as described herein, can be readily identified by those having familiarity with the art in view of the instant disclosure.

According to certain embodiments, SARPs may be extracted in a solvent comprising at least 25% isopropanol (typically aqueous isopropanol), and in certain embodiments such a solvent may comprise at least 25,30, 35, 40, 45, 50, 55, 60, 65, 70, 75, 80, 85, 90 or 95% isopropanol. In one embodiment SARP is extracted in a solvent comprising isopropanol: acetone: toluene at a ratio of 1:2:1, and in another embodiment the protein is extracted in toluene. In these and related embodiments, SARP may be extracted from a biological sample in a solvent as provided herein to obtain a soluble extract free from insoluble material, which soluble extract may be achieved by any of a number of methodologies for separating soluble from insoluble materials as are well known to the art, including, for example, centrifugation, filtration, precipitation, evaporation, condensation, distillation, or the like.

Once obtained, the soluble extract, containing the solubilized SARP, and being free from insoluble material, may be subjected to a step in which SARP is resolved from the extract, i.e., whereby SARP may be separated from one or more other components that may be present in the soluble extract. As described in greater detail in the Examples, SARP may in certain particularly preferred embodiments be resolved from the soluble extract using a reversed-phase separations, which may include any molecular separation technique in which, as a function of the solution conditions that are present, relatively non-polar to moderately polar solute molecules can be removed from a relatively polar environment to a relatively non-polar environment.

Preferred examples of reversed-phase separations may therefore include reversed-phased chromatography procedures such as RP-HPLC, reversed-phase solid-phase extraction, ion-pairing chromatography, phase partitioning, partition chromatography, or the like, whereby distinctive hydrodynamic and/or physicochemical properties of SARPs and of other solutes that may be present in the soluble extract can be differentially exploited to effect resolution (i.e., physical separation) of SARP from the extract. For instance, and as described in greater detail below, RP-HPLC separation of SARP may be effected using a C₄ stationary phase and an acetonitrile-isopropanol gradient in trifluoroacetic acid (TFA) aqueous solutions. Other suitable stationary phases may vary as a function of the solvent system and particle size employed and might include, for instance, C₅ C₈, C₁₂, C₁₈, phenyl, polystyrene-divinylbenzene, or the like. These and other reversed-phase separation modalities are known to those familiar with the art and can be combined with additional methodologies for determining the presence of a SARP according to the disclosure provided herein.

### 3. Conformers

SARP of the present invention exhibit unusual molecular flexibility. Hence, in certain preferred embodiments such flexibiliy comprises at least two global conformers and several sub-states conformers. Proteins that exhibit rich morphologies are usually disordered proteins with several partially folded states (Tompa (2002) Trends Biochem Sci 27:527. In humans may neurodegenerative diseases are caused by accumulation of intrinsically unstructured proteins. The osmiophilic deposits (3, 4) observed in the Sauromatum appendix may represent aggregates of the expanded states (state A) of SARP. Many studies have demonstrated that IUPs exist without a well-defined folded structure in vitro. The lack of a well-defined structure may allow binding to many different ligands using different conformational states. This also allows a rapid association and dissociation of complexes for intracellular regulatory network. Intrinsically unstructured proteins functions are divided into 6 categories (43) and SARP may fulfill at least two categories, assemblers and scavengers. Self-assembly is clearly evident from the micrographs and scavenger activity is attractive because it may explained why it is heavily stained with osmium. It may store lipids or other small ligands and this association enables its purification in organic solution.

As described in greater detail below, SARP conformers of the invention has been characterized with respect to it CSD using ESI/MS. In this context, “˜” will be understood to encompass deviations from the apparent CSD of 34.1 kDa by no more than ten %, preferably no more than five %, more preferably no more than three %, more preferably no more than two %, more preferably no more than one %, and still more preferably by no more than one-half of one %, which deviations may, according to non-limiting theory, result from any one or more of potential technical limitations of the instrumentation, methodology and/or technique used to characterize SARP conformers as a function of the particular subject or biological source from which it is isolated, or from presently unrecognized unusual attributes of the protein that influence its behavior when subjected to the characterization conditions described herein. Accordingly, those familiar with the art will be able readily to determine whether a particular SA related-compound altered (e.g., increased or decreased in a statistically significant manner) the conformation state of SARP.

### 4. Ligands and Ligand-Binding Sites

SARP of the present invention at least one binding site for a ligand that is selected from a salicylate-related compound as provided herein, and ATP or GTP, and NAD(P) or NAD(P)H. Such a binding site may therefore be any naturally occurring binding interaction structure by which a cognate ligand specifically binds to, or associates with SARP. In certain preferred embodiments SARP, or a fragment or a variant thereof, may comprise a binding site for a salicylate-related compound that is SA, ASA or 2,6-DHBA. In certain other preferred embodiments SARP (or a fragment or a variant thereof) or an assembled SARP structure may comprise at least two binding sites for a ligand that is selected from a salicylate-related compound, a nucleotide triphosphate, and nicotinamide adenine dinucleotide. Ligands that are capable of specifically binding may bind a SARP molecule with an affinity constant, Kₐ, of greater than or equal to ˜10⁵ to 10⁷ M⁻¹, preferably of greater than or equal to ˜10⁸ M⁻¹, more preferably of greater than or equal to ˜10⁹ M⁻¹ and still more preferably of greater than or equal to ˜10¹⁰ M⁻¹. In certain embodiments binding affinity constants of ligands for SARP may exceed 10¹¹ to 10¹² M⁻¹. Affinities of SARP molecules for ligands according to the present invention can be readily determined using conventional techniques, for example those described by Scatchard et al. (1949 Ann. N.Y. Acad. Sci. 51:660), or by techniques described herein (see, e.g., the Examples).

### 5. Salicylate-Related Compounds

Salicylate-related compounds include SA and its derivatives, ASA and its derivatives, and 2,6-DHBA and its derivatives, including salts and derivatives such as sodium salicylate, or O-acetylsalicyloyl chloride, sodium thiosalicylate (Thiocyl), salsalate (Disalcid), diflunisal (Dolobid), triethanolamine salicylate (Trolamine), octyl salicylate, homomethyl salicylate, and other salicylate structural analogues, precursors, prodrugs, metabolites and degradation products which preferably substantially retain a BA or similar core structure (see, e.g., Goodman and Gilman, 2001 In: The Pharmacological Basis of Therapeutics 10^{th} ed., Hardman and Limbird, eds., McGraw-Hill, NY; Brune, 2002 Am. J. Ther. 9:215; Paterson et al., 2001 Q. J. Med. 94:445; Furst, 1994 Arth. Rheum. 37:1; Vane et al., 1987 FASEB J. 1:89; Needs et al., 1985 Clin. Pharmacokinet. 10:164). Methods for detecting the presence of a salicylate-related compound are described herein using SARP, and other methods are also known to the art (e.g., Stewart et al., 1987 Ann. Clin. Biochem. 24:552). Particularly preferred salicylate-related compounds according to the present invention may be ligands for the herein described SARPs by virtue of their ability to bind the protein specifically; these include SA, ASA, 2,6-DHBA, BA, 2,1,3-benzothiadiazole, and 3,4-dihydroxybenzoic acid.

### 6. UV Absorbance

As disclosed herein, SARPs also exhibit UV absorbance spectra that are nonlinear and which feature one or more in the presence of a salicylate-related compound as described herein, and which lack any such non-linearity when a salicylate-related compound is not present. According to non-limiting theory, the nonlinearity may signify coupling and electron transfer between different SARP confomers. For example, a first SARP confomer can bind to a second SARP confomer via SA that have one valence electron. The pK of SA is 3 and it is in the form of an anion in water, such that further according to theory, SA is capable of accepting a proton or delivering an electron to SARP.

Additionally and as described herein, SARPs are capable of unidirectionally transferring one or two electrons to an electron acceptor such as 3-(4,5-dimethyl thiazol-2-yl-(2,5-diphenyl) tetrazolium bromide (MTT), nitro blue tetrazolium (NBT) or neotetrazolium chloride (Neo-NBT), or to ferricytochrome c.

Thus, according to certain related embodiments, UV absorbance spectra of SARP may fluctuate over time when absorbance measurements are collected over a range of wavelengths and at a plurality of time points, if a salicylate-related compound is present or, under other chemical environments as described below. Specifically and in certain preferred embodiments, an absorbance spectrum of SARP at wavelengths 190 nm to 280 nm in the absence of a salicylate-related compound comprises no detectable absorbance, but in the presence of a salicylate-related compound the absorbance spectrum of the protein comprises a change in the UV absorbance. In certain preferred embodiments the change may be detected at wavelengths from (inclusively) 190 nm to 280 nm, where it will be appreciated that selection of a suitable wavelength may vary as a function of several factors including protein concentration, solution composition (e.g., absorbance properties of buffers, salts, etc.), instrument sensitivity, and other related attributes of a spectroscopic configuration. Hence, the occurrence of a change in the absorbance spectrum of SARP in the presence of a salicylate-related compound, and the absence of such change in the spectrum when no salicylate-related compound is present, provide a means for identifying and/or characterizing SARPs, and/or for probing the structures of transient SARP product states over time.

### 7. Electron Transfer

Another distinguishing property of SARP described herein is its ability to function as a redox protein, that is, to transfer at least one electron to a suitable electron acceptor molecule under certain defined solution conditions. Under certain such conditions described herein SARP is capable of transferring two electrons to a suitable electron acceptor. A number of molecular species are capable of acting as electron donors (i.e., as reducing agents) and/or as electron acceptors (i.e., as oxidizing agents) (see, e.g., Mayor et al., 2002 Ann. N.Y. Acad. Sci. 960:16; Carroll et al., 2002 Angew. Chem. Int. Ed. 41:4378), including reduction/oxidation or “redox” proteins which mediate such electron transfer (Bakhshi, 1994 Prog. Biophys. Molec. Biol., 61:187; Gilardi et al., 2001 Trends Biotechnol. 19:468; Gray and Winker, 1996 Ann. Rev. Biochem. 65:537; Marcus and Sutin, 1985 Biochim. Biophys. Acta 811:265). Redox proteins typically comprise one or more redox centers, specialized sites that by virtue of their structure render the protein capable of mediating electron transfer.

Electron acceptors for use with SARP of the present invention include those compositions that are capable of accepting and donating electrons from SARP as evidenced by art-accepted methodologies, such as altered (i.e., increased or decreased in a statistically significant manner) conductivity, chemical reactivity and/or spectral properties (e.g., color, absorbance or optical density), or other parameters known for a particular candidate electron acceptor to be indicative of an altered redox state. Particularly preferred electron acceptors for SARP under conditions described herein are tetrazolium salts [3-(4,5-dimethyl thiazol-2-yl-(2,5-diphenyl) tetrazolium bromide (MTT), nitro blue tetrazolium (NBT), neotetrazolium chloride (Neo-NBT), and ferricytochrom c.

### 8. Molecular Assembly

Determination of the relative ability of a protein or polypeptide to oligomerize is well within the knowledge of the relevant art, where any of a number of established methodologies may be applied to detect protein oligomerization, or formation of a protein complex, including covalent or non-covalent protein-protein associations such as dimerization, trimerization, etc. or other degrees of multimer or oligomer formation as may yield such a complex of associated protein molecules (see, e.g., Scopes, Protein Purification: Principles and Practice, 1987 Springer-Verlag, New York). For example, biochemical separation techniques for resolving proteins on the basis of molecular size (e.g., gel electrophoresis, gel filtration chromatography, analytical ultracentrifugation, etc.), and/or comparison of protein physicochemical properties, for instance, molecular or supramolecular sizing by light-scattering techniques (e.g., dynamic light scattering such as laser diffraction), by photon correlation spectroscopy or by phase Doppler velocimetry, or characterization before and after introduction of sulfhydryl-active (e.g., iodoacetamide, N-ethylmaleimide) or disulfide-reducing (e.g., 2-mercaptoethanol, dithiothreitol) agents, or other equivalent methodologies, may all be employed for determining a degree of polypeptide dimerization or oligomerization, and for determining possible contribution of specific structural features such as disulfide bonds to such potential quarternary structure.

Accordingly, those familiar with the art will be able readily to determine whether a particular SA related-compound altered (e.g., increased or decreased in a statistically significant manner) the ability to oligomerize. For instance, the resemblance of SARP structures to dendrimer structures (Hedden and Bauer, 2003 Macromol. 36:1829; Young-Soo, et., 2002 Langmuir, 18:5927) may be examined by light-scattering and small-angle neutron scattering techniques, photon correlation spectroscopy, phase Doppler velocimetry or, characterization before and after introduction of sulfhydryl-active (e.g., iodoacetamide, N-ethylmaleimide) or disulfide-reducing (e.g., 2-mercaptoethanol, dithiothreitol) agents, or by changing other functional group such as transition metals or other non-proteinaceous, redox moieties, or other equivalent methodologies, which may all be employed for determining a degree of polypeptide dimerization or oligomerization, and for determining possible contribution of specific structural features such as disulfide bonds and other functional groups to such potential quarternary structure. Accordingly, those familiar with the art will be able readily to determine whether a particular SARP displays altered (e.g., increased or decreased in a statistically significant manner) ability to oligomerize.

Certain SARP properties described in greater detail below relate to SARP protein complexes that may be present in one of several oligomeric states depending upon the solution conditions (e.g., SARP concentration, pH, solvent strength, solvent polarity/polarizability, nature and amount of other solutes, temperature, presence or absence of SA, etc.).

### 9. SARPs in Nanotechnology

SARPs can be detected optically and chemically; they can transfer electrons; they are capable of self-assembly; and their dimensions are in the nanoscale. These properties make them candidates, in certain embodiments, for nanotechnological applications (Gilardi and Fantuzzi, 2001 Trends Biotech. 19:468). Certain Nanotechnology applications include any of a variety of situations where it may be desirable to exploit controllable assembly of oligomeric SARPs in spatially defined structures, typically organized into supramolecular ensembles at a nanoscopic (nanometer) or even microscopic (micrometer) scale (e.g., Liu et al., 2002 Proc. Nat. Acad. Sci. USA 99:5165, and references cited therein) as may be suitable for a particular use. Generation and characterization of assembled oligomers of SARPs are described herein, including discrete structural organizing motifs such as membrane-like films, or branched or elongated tube-like polymeric structures that result from exposure of SARPs to different chemical environments (e.g., those that affect SARP redox-states). Liu et al. (2002) also describe instrumentation and methodologies for directing the positioning of such supramolecular assemblies. Determination of the properties of such assembled SARP oligomers, for instance, characterization of the size, shape, orientation on a solid surface, optical, electrical conductivity and/or redox functionalities such as those described herein, and the like, may be performed according to art-established methodologies. For example, Yeates et al., 2002 Curr. Opin. Struct. Biol. 12:464, summarize naturally occurring proteins that are capable of assembly into linear, filamentous and/or tubular supramolecular structures, while Vijayamohanan et al., 2001 Appl. Biochem. Biotechnol. 96:25, disclose methodological considerations pertaining to substrate properties, formation and electrical conductivity properties of self-assembling monolayers prepared from non-protein biomolecules. Also pertinent to these and related embodiments will be techniques such as those described in Gilardi et al. (Trends Biotechnol. 2001 19:468), Liu et al. (2002), and Bakhshi (Prog. Biophys. Molec. Biol. 1994 61:187).

### 10. Beating Gels

SARP in the presence of SA forms, under appropriate conditions, a polymer gel that can oscillate for several hours. The gel may undergo an autonomous swelling-deswelling oscillation without alteration of the external conditions. SARP is thus capable of mechanical oscillation when assembled into a polymer network, resulting in a stimulus-responsive, self-oscillating gel which autonomously exhibits cyclic swelling-deswelling motion under constant conditions. The invention therefore contemplates use of this “smart gel” for drug delivery (Leroux, 1999 Chaos 9:267), hydrogels (Yoshida et al., 1999 Chaos 9:260; 2003 Macromol. 36:1759, 2002 J. Am, Chem. Soc. A104:7549), and cell culture.

In certain preferred embodiments SARP is present as a gel, which includes a protein complex that is capable of assembly in a concentration-independent fashion, preferably in water and in the absence of added dissolved salts. A protein complex comprising a SARP gel is typically viscous, such that it excludes entrapped interstitial liquid when subjected to appropriate physical forces such as excessive gravitational forces produced by centrifugation (e.g., at least 10×g, 100×g, 1,000×g, 10,000×g, 25,000×g, 50,000×g, 70,000×g, 100,000×g, or more). Other physicochemical methodologies for determining viscosity are also known to the art, for example, by using a falling-ball viscometer or other means for determining viscosity of a material. By these criteria a gel may be differentiated from an aggregate, which refers to a non-gel protein complex that forms as a function of protein concentration.

The condensation and relaxation in a periodic manner is characteristic of assembled SARP polypeptides under certain conditions, as described herein. For example, an oscillating SARP gel as provided herein includes any SARP protein complex in gel form wherein a repeating change over time in at least one dimensional state is detectable over a measurable unit of time, with statistical significance, using methodologies described herein and known to the art. Without wishing to be bound by theory, it is believed that the energy for the mechanical oscillation may be generated by the recycling of electrons between SARP conformations. Energy release is well known in reactions involving free radicals.

Certain embodiments of the invention therefore contemplate use of SARPs to form a stimulus-responsive smart hydrogel. SARP behavior can lead to the development of gel-based actuators, valves, sensors, controlled-release systems for drugs and other substances, artificial muscles for robotic devices, chemical memories, optical shutters, molecular separation systems, and toys. Other potential applications that can be considered include paints, coatings, adhesives, recyclable absorbents, bioreactors containing immobilized enzymes, bioassay systems, and display devices.For example, recent studies have shown that certain drugs are better administered in a periodic, pulsed manner. Accordingly, certain embodiments relate to SARP assembly into a self-oscillating gel, in the presence of a desired drug. As disclosed herein, SARP has a high affinity for low molecular weight lipidic molecules. Without wishing to be bound by theory, such affinity may reflect spatial organization in SARP of charged moieties in a manner that permits SARP to be used as an ion trap for low molecular weight drugs such as insulin, chemotherapeutic agents, or other desired drug molecules. SARP gel may be implanted underneath the skin or close to the desired target tissue. Administration and uptake of SA may then be employed to start the gel oscillation, permitting the drug to be released. Determination of the loading capacity of SARP gel, the rate of release and the stability of the gel in the human body can all be performed according to art-accepted techniques. For example, well established methods for studying the kinetics of pulsatile drug release may be employed (Marszalek et al., 1997 Biophys. J. 73:1169; Kikuchiand and Okano, 2002 Adv. Drug Del. Rev. 54:53).

### 11. Biological Samples

Biological samples may be provided by obtaining a sample of xylem, phloem or sap, a blood sample, biopsy specimen, tissue explant, organ culture, biological fluid or any other tissue or cell preparation from a subject or a biological source. The subject or biological source may be a plant (e.g., a thermogenic plant as described herein), a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines and the like.

In certain preferred embodiments the biological sample comprises at least one cell from a subject or biological source, and in certain other preferred embodiments the biological sample is a biological fluid containing a SARP. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include blood, serum and serosal fluids, plasma, lymph, urine, cerebrospinal fluid, saliva, mucosal secretions of the secretory tissues and organs, ascites fluids such as those associated with non-solid tumors, fluids of the pleural, pericardial, peritoneal, abdominal and other body cavities, and the like. Biological fluids may also include liquid solutions contacted with a subject or biological source, for example, cell and tissue culture medium including cell or tissue conditioned medium, elutriation or lavage fluids and the like.

By way of background, and as a non-limiting illustrative example of a thermogenic plant, S. guttatum that undergoes a dramatic metabolic change on “D-day”, the first day of flowering. This metabolic outburst is triggered by SA, which originates in the male flowers and moves to the appendix on the eve of heat-production (Raskin et al., 1987 Science 237:1601); ASA and 2,6-DHBA can also trigger heat production. On the day before flowering, the level of SA leaps nearly 100-fold in the appendix, a sterile, finger-shaped structure found at the upper end of the inflorescence (Raskin et al., 1989 Proc. Natl. Acad. Sci. USA 86:2214). Consequently, the metabolic rate within the inflorescence increases sharply, and its internal temperature may rise to as high as 32° C. On D-day, the appendix displays a striking elevation of respiration rate, pronounced heat production evidenced by warming of the tissue, and exhalation of a strong foul odor upon opening of the inflorescence (Meeuse, 1985, In: The physiology and biochemistry of plant respiration, Palmer J M (ed.), Cambridge University Press, Cambridge, UK, pp. 47-58; Skubatz et al., 1995 Proc. Nat Acad. Sci. USA 92:1084).

The heat generated by the Sauromatum appendix apparently serves as a volatilizer for certain odoriferous compounds which attract pollinators (Skubatz et al., 1995 Proc. Nat. Acad. Sci. USA 92:1084; Skubatz et al., 1996 New Phytol. 134:631, Skubatz et al., 1999 Am. J. Bot. 86:841). Oxygen consumption by the mitochondria of the appendix tissue is remarkably insensitive to cyanide; mitochondrial heat production occurs via an alternative pathway (Moore et al., 1991 Biochim. Biophys. Acta 1059:121; Skubatz et al., 1992 Biochim. Biophys. Acta 1100:98; Skubatz et al., 2000 J. Electron Micros. 49:775; Skubatz et al., 2001 Flora 196:446; Vanlerberghe et al., 1997 Annu. Rev. Plant Physiol. Plant Mol. Biol. 48:703). The oxidase responsible for the oxidation of the reduced ubiquinone pool in the mitochondria has been isolated from the Sauromatum mitochondria and is ubiquitous in plants. The large amount of starch present in the appendix tissue is the fuel for this thermogenic, wasteful respiration.

The appendices of some other species such as Arum Italicum, Amorphophallus konjac, Dracunculus vulgaris (Araceae), and also appendages of Victoria cruziana flowers (Nymphaeaceae), and sporophylls of male cones of Encephalartos ferox (Cycadaceae), have high rates of respiration that lead to an appreciable rise in their temperature above the ambient (Meeuse et al., 1988 Sex. Plant Reprod. 1:3; Skubatz et al., 1990 Planta 182:432, 1991 Plant Physiol. 95:1084). SA was detected in these thermogenic organs at levels sufficient to trigger heat-production, as well as in non-thermogenic plants (Raskin et al., 1990 Annals. Bot. 66:369).

### 12. Salicylate Biological Effects

Certain embodiments of the present invention relate to a “salicylate biological effect”, which includes any metabolic process, pathologic or physiologic state, disease, disorder, condition, syndrome, or the like, wherein at least one deviation or departure from a physiological norm (e.g., temperature change, metabolic or respiratory rate, concentration of a physiological solute, etc.) causes, correlates with, is accompanied by or results from an alteration (i.e., a statistically significant change), whether appropriate (e.g., desirable) or inappropriate, to the structure, activity, function, expression level, physicochemical or hydrodynamic property, or stability of a molecular component of a biological pathway that comprises a SARP molecule as provided herein (e.g., a biological signal transduction pathway), or in structural or functional changes that occur as the result of interactions between intermediates that may be formed as the result of such interactions, including metabolites, catabolites, substrates, precursors, cofactors and the like, wherein typically such alteration results, for example, from a statistically significant change in the level of a salicylate-related compound that is present. In preferred embodiments the molecular component of a biological pathway such as a biological signal transduction pathway that comprises a SARP may be a protein, peptide or polypeptide, and in certain other preferred embodiments the component may be an amino acid, or derivative thereof; a lipid, fatty acid or the like, or derivative thereof; a carbohydrate, saccharide or the like, or derivative thereof, a nucleic acid, nucleotide, nucleoside, purine, pyrimidine or related molecule, or derivative thereof, or the like; an alkaloid; a phenolic or aromatic compound; or any covalently or non-covalently complexed combination of these components, or any other biological molecule.

Persons skilled in the art will be familiar with an array of criteria according to which it may be recognized what are, e.g., biological, physiological, pathological and/or clinical signs and/or symptoms of salicylate biological effects as provided herein, for example, the effects of SA described above and including without limitation SA effects on plant pathogen resistance, plant resistance to frost damage, malignant cell growth rates, decreased interaction with extracellular matrix, an altered oxygen level in a cell, inflammation, neurological conditions, skin disorders and dermatological conditions, cancer, cell cycle regulation, cell survival, apoptosis, metabolic status including diabetes, obesity and related disorders, allergy, and undesirable drug side-effects.

As noted above, SA influences specific pathways for biological signal transduction, including those associated with analgesic, antipyretic, antirheumatic, anti-cancer, anti-diabetic, keratolytic and anti-inflammatory effects, cell division, cell survival, apoptosis, cellular proliferation and differentiation, and plant disease resistance. Accordingly, “biological signal transduction pathways” or “inducible signaling pathways” in the context of the present invention include transient or stable associations or interactions among molecular components involved in the control of these and similar processes in cells. Depending on the particular pathway of interest, an appropriate parameter for determining induction of such pathway may be selected. For example, for signaling pathways associated with cell proliferation, there is available a variety of well known methodologies for quantifying proliferation, including, for example, incorporation of tritiated thymidine into cellular DMA, monitoring of detectable (e.g., fluorimetric or colorimetric) indicators of cellular respiratory activity, or cell counting, or the like. Similarly, in the cell biology arts there are known multiple techniques for assessing cell survival (e.g., vital dyes, metabolic indicators, etc.) and for determining apoptosis (e.g., annexin V binding, DMA fragmentation assays, caspase activation, etc.). Other signaling pathways will be associated with particular cellular phenotypes, for example specific induction of gene expression (e.g., detectable as transcription or translation products, or by bioassays of such products, or as nuclear localization of cytoplasmic factors), altered (e.g., statistically significant increases or decreases) levels of intracellular mediators (e.g., SA or a salicylate-related compound, or their metabolites, activated kinases or phosphatases, altered levels of cyclic nucleotides or of physiologically active ionic species, etc.), or altered cellular morphology, and the like, such that cellular responsiveness to a particular stimulus as provided herein can be readily identified to determine whether a particular cell comprises an inducible signaling pathway.

In the context of SA effects in cancer, malignancy, neoplasia, metastasis, and the like, the presence of a malignant condition in a subject refers to the presence of dysplastic, cancerous and/or transformed cells in the subject, including, for example neoplastic, tumor, non-contact inhibited or oncogenically transformed cells, or the like (e.g., adenoma, epithelioma, colorectal cancer, leukemia, lymphoma, melanoma, carcinomas such as adenocarcinoma, squamous cell carcinoma, small cell carcinoma, oat cell carcinoma, etc., sarcomas such as chondrosarcoma, osteosarcoma, etc.) which are known to the art and for which criteria for diagnosis and classification are established. Many cancer cells exhibit statistically significant decreases in their ability to interact with (e.g., bind or respond to) extracellular matrix; certain cancer cells may respire only anaerobically. In certain embodiments such cancer cells are malignant hematopoietic cells, such as transformed cells of lymphoid or myeloid lineages and the like; cancer cells may in certain preferred embodiments also be epithelial cells such as carcinoma cells.

### 13. Antibodies to SARPs

Within an aspect of the invention, isolated SARP, and peptides or peptide fragments derived from isolated SARP (e.g., products of chemical or enzymatic cleavage of isolated SARP, such as with trypsin, chymotrypsin, pepsin, papain, Staphylococcus V8 protease or other proteases, or with CNBr, formic acid, concentrated HC1 or other chemical agents for cleaving proteins), may be utilized to prepare antibodies that specifically bind to SARP. Peptides or peptide fragments of SARP preferably comprise at least 4, 5, 6, 7, 8, 9, 10, 11, 12, 13, 14, 15, 16, 17, 18, 19, 20, 21-25, 26-30, 31-35 or more contiguous amino acids from the isolated protein, for example, any of the products of tryptic digestion of the isolated protein as depicted in Table VI. The term “antibodies” is meant to include polyclonal antibodies, monoclonal antibodies, fragments thereof such as F(ab′)₂, and Fab fragments, as well as any recombinantly produced binding-partners. Antibodies are defined to be specifically binding if they bind a SARP molecule with a Kₐ of greater than or equal to ˜10⁴ M⁻¹, preferably ˜10⁵ M⁻¹, more preferably ˜10⁶ M⁻¹ and still more preferably ˜10⁷ M⁻¹. Affinities of binding-partners or antibodies can be readily determined using conventional techniques, for example those described by Scatchard et al., (Ann. N.Y. Acad. Sci., 1949 51:660) or, by surface plasmon resonance (Wolff et al., 1993 Cancer Res. 53:2560; BlAcore/Biosensor, Piscataway, N.J.).

Polyclonal antibodies can be readily generated from a variety of sources, for example, horses, cows, goats, sheep, dogs, chickens, rabbits, mice or rats, using procedures that are well known in the art. In general, isolated SARP, or a peptide derived therefrom that is appropriately conjugated, is administered to the host animal typically through parenteral injection. The immunogenicity of SARP molecule or peptide may be enhanced through the use of an adjuvant, for example, Freund's complete or incomplete adjuvant. Following booster immunizations, small samples of serum are collected and tested for reactivity to SARP molecules or SARP-derived peptides. Examples of various assays useful for such determination include those described in Antibodies: A Laboratory Manual, Harlow and Lane (eds.), Cold Spring Harbor Laboratory Press, 1988; as well as procedures such as countercurrent immuno-electrophoresis (CIEP), radioimmunoassay (RIA), radioimmunoprecipitation, enzyme-linked immunosorbent assays (ELISA), dot blot assays, and sandwich assays, see, e.g., U.S. Pat. Nos. 4,376,110 and 4,486,530, or other similar assays known in the art.

Monoclonal antibodies specific for a desired protein antigen (such as the isolated SARP described herein) may be readily prepared using well-known procedures, see for example, the procedures described in Current Protocols in Immunology (Wiley & Sons, NY, Coligan et al., eds., 1994; U.S. Pat. Nos. RE 32,011, 4,902,614, 4,543,439 and 4,411,993; Monoclonal Antibodies, Hybridomas: A New Dimension in Biological Analyses, 1980, Plenum Press, Kennett et al., eds.). Briefly, the host animals, such as mice are injected intraperitoneally at least once, and preferably at least twice at ˜three-week intervals with isolated and purified SARP or conjugated SARP protein peptide, optionally in the presence of adjuvant. Mouse sera are then assayed by conventional dot blot technique or antibody capture (ABC) to determine which animal is most suitable as a source of splenocytes for fusion to a myeloma partner cell line. Approximately two to three weeks later, the mice are given an intravenous boost of SARP molecule or peptide. Mice are later sacrificed and spleen cells fused with commercially available myeloma cells, such as Ag8.653 (ATCC), following established protocols. Briefly, the myeloma cells are washed several times in media and fused to mouse spleen cells at a ratio of ˜three spleen cells to one myeloma cell. The fusing agent can be any suitable agent used in the art, for example, polyethylene glycol (PEG). The cell suspension containing fused cells is plated out into plates containing media that allows for the selective growth of the fused cells. The fused cells can then be allowed to grow for approximately eight days. Supernatants from resultant hybridomas are collected and added to a plate that is first coated with goat anti-mouse lg. Following washes, a label, such as, ¹²⁵I-SARP is added to each well followed by incubation. Positive wells can be subsequently detected by autoradiography. Positive clones can be grown in bulk culture and supernatants are subsequently purified over a Protein A column (Pharmacia).

The monoclonal antibodies of the invention can be produced using alternative techniques, such as those described by Alting-Mees et al., 1990 (Monoclonal Antibody Expression Libraries: A Rapid Alternative to Hybridomas, Strategies Mol. Biol. 3:1-9). Similarly, binding partners can be constructed using recombinant DNA techniques to incorporate the variable regions of a gene that encodes a specific binding antibody. Such a technique is described by Larrick et al., 1989 Biotechnol. 7:394.

Other types of “antibodies” may be produced using the information provided herein in conjunction with the state of knowledge in the art. For example, antibodies that have been engineered to contain elements of human antibodies that are capable of specifically binding SARP are also encompassed by the invention. An additional method for selecting antibodies that specifically bind to a SARP or fragment thereof is by phage display, e.g., Winter et al., 1994 Annu. Rev. Immunol. 12:433; Burton et al., 1994 Adv. Immunol. 57:191. Human or murine immunoglobulin variable region gene combinatorial libraries may be created in phage vectors that can be screened to select lg fragments (Fab, Fv, sFv, or multimers thereof) that bind specifically to a SARP molecule or fragment thereof. See, e.g., U.S. Pat. No. 5,223,409; Huse et al., 1989 Science 246:1275; Kang et al., 1991 Proc. Natl. Acad. Sci. USA 88:4363; Hoogenboom et al., 1992 J. Molec. Biol. 227:381; Schlebusch et al., 1997 Hybridoma 16:47, and references cited therein. For example, a library containing a plurality of polynucleotide sequences encoding lg variable region fragments may be inserted into the genome of a filamentous bacteriophage, such as M13 or a variant thereof, in frame with the sequence encoding a phage coat protein, for instance, gene III or gene VIII of M13, to create an M13 fusion protein. A fusion protein may be a fusion of the coat protein with the light chain variable region domain and/or with the heavy chain variable region domain. Once isolated and purified, the antibodies against SARP may be used to detect the presence of SARP in a sample using established assay protocols. Further, the antibodies of the invention may be used therapeutically to bind to SARP and alter its activity in vivo.

As provided herein and according to methodologies well known in the art, polyclonal and monoclonal antibodies may be used for the affinity isolation of SARPs. See, e.g., Hermanson et al., 1992 (Immobilized Affinity Ligand Techniques, Academic Press, Inc. NY). Briefly, an antibody (or antigen-binding fragment thereof) may be immobilized on a solid support material, which is then contacted with a sample comprising the polypeptide of interest (e.g., SARP molecules). Following separation from the remainder of the sample, SARP is then released from the immobilized antibody.

### 14. Screening Assays for Modulators of SARP

The present invention provides compositions, methods and kits for use in identifying agents that alter a salicylate biological effect as provided herein. Such screens and assays are designed to detect an effect of a candidate agent on the binding between a SARP molecule and a SARP ligand, for example, a salicylate-related compound, ATP or GTP, and NAD(P)/NAD(P)H.

As used herein, the term “screening” refers to the use of assays designed to identify agents that alter (e.g., increase or decrease in a statistically significant manner) a biological effect. Briefly, in certain embodiments, SARPs are contacted with a known ligand in the absence and presence of a candidate agent and under conditions and for a time sufficient for ligand binding to the protein to occur (if in the absence of the agent), and the effect of the agent on the binding interaction between the ligand and SARP is determined. According to certain other embodiments, SARPs are contacted with a known electron acceptor in the absence and presence of a candidate agent and under conditions and for a time sufficient for electron transfer to the electron acceptor to occur (e.g., if in the absence of the agent), and the effect of the agent on CSD and electron transfer from SARP to the acceptor are determined. According to certain embodiments CSD is determined in the absence and presence of a candidate agent. According to certain other embodiments, absorbance by SARPs of UV light at one or a plurality of wavelengths within a wavelength range of 190-300 nm (and in certain preferred embodiments 190-230 nm, 190-250 nm, 190-215 nm or 190-280 nm), and at one or a plurality of time points, is determined in the absence and presence of a candidate agent to determine whether the conformational state of SARP is altered (e.g., enhanced or depressed in a statistically significant manner at one or more protein ions and wavelengths and/or at one or more time points).

A candidate agent may alter any of the herein described parameters (e.g., ligand binding, electron transfer, UV absorbance, or light scattering) directly (e.g., by physical contact with SARP at a site of ligand binding, electron transfer, UV absorbance, or light scattering) or indirectly (e.g., by interaction with one or more proximal or distal sites within the protein, as may according to non-limiting theory alter the described parameter by interacting with other than a site of ligand binding, electron transfer, UV absorbance or light scattering, for instance, by changing the conformation of SARP. In some embodiments, the candidate agent may be a peptide, polypeptide, protein or small molecules, and in certain preferred embodiments the candidate agent may be a salicylate-related compound as provided herein, or a structural mimetic of a salicylate-related compound. Typically, and in more preferred embodiments such as for high throughput screening, candidate agents are provided as “libraries” or collections of compounds, compositions or molecules. Such molecules typically include compounds known in the art as “small molecules” and having molecular weights less than 10⁵ s, preferably less than 10⁴ s and still more preferably less than 10³ s. For example, members of a library of test compounds can be administered to a plurality of samples, each containing at least one SARP as provided herein (or a SARP variant or a plant reductase as described herein), and then assayed for their ability to alter at least one of the herein-described parameters.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies arid recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694 and PCT/US91/04666) or other compositions that may include small molecules as provided herein (see e.g., PCT/US94/08542, EP 0774464, U.S. Pat. No. 5,798,035, U.S. Pat. No. 5,789,172, U.S. Pat. No. 5,751,629). Those having ordinary skill in the art will appreciate that a diverse assortment of such libraries may be prepared according to established procedures, and tested using a SARP target such as a S. guttatum polypeptide or homolog according to the present disclosure.

There are a variety of assay formats known to those of ordinary skill in the art for detecting binding interactions between proteins and their cognate iigands. See, e.g., Harlow and Lane, 1988 In: Antibodies: A Laboratory Manual, Cold Spring Harbor Laboratory. Within one embodiment, a protein or polypeptide is immobilized on a solid support prior to contact with the ligand. Binding may then be detected using a detection reagent that specifically binds to the protein, for example, at a site known or suspected of being a site of ligand interaction (e.g., an antibody or fragment thereof), or using a detectable portion of the protein (e.g., direct detection of a UV-absorbing moiety, or detection of electron transfer to an acceptor molecule, or detection of SARP molecular assembly state such as assembled SARP swelling/de-swelling).

A solid support may be any material known to those of ordinary skill in the art. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. A polypeptide may be immobilized on the solid support using a variety of techniques known to those of skill in the art, which are amply described in the patent and scientific literature. In the context of the present invention, the term “immobilization” refers to both non-covalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time.

Binding is generally allowed to occur under solution conditions and for an amount of time sufficient to detect the bound ligand. An appropriate amount of time may generally be determined by assaying the level of binding that occurs over a period of time. After incubating under conditions and for a time sufficient to permit interaction of SARP, ligand, and candidate agent, the level of ligand-SARP binding is detected and compared to the level of binding in the absence of the agent.

For example, following a suitable interval for ligand binding, unbound ligand is removed, and bound ligand is detected using a linked reporter group or a separate detectable marker comprising a reporter group. The method employed for detecting binding depends upon the nature of the reporter group employed. When electron transfer is detected, fluorescence or colorimetric or other techniques may be used. For radiometric quantification of ligand binding (or, e.g., competitive inhibition by a candidate agent of the binding to SARP of a detectably labeled ligand comprising a radioactive group), scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

An agent that binds to SARP and/or to SARP/ligand complexes may result in a detectable decrease or increase in SARP-ligand binding. Such altered levels of ligand binding pertain to readily detectable increases or decreases that may vary quantitatively depending on the proteins being monitored and on the particular reagents, instrumentation and methodology selected. An altered level of ligand-SARP binding refers to a statistically significant increase or decrease.

### 15. Pharmaceutical Compositions

Clinically beneficial agents, including therapeutic and/or diagnostic agents identified according to the methods described herein, and also in certain embodiments including the present invention SARP, for example, in the form of a protein complex comprising a plurality of SARP molecules in an oligomeric state, for biological delivery of an agent, may be formulated into pharmaceutical compositions for administration according to well known methodologies, typically in combination with a pharmaceutically acceptable carrier, excipient or diluent. Such carriers will be nontoxic to recipients at the dosages and concentrations employed. For therapeutic agents, ˜0.01 μg/kg to ˜100 mg/kg body weight will be administered, typically by the intradermal, subcutaneous, intramuscular or intravenous route, or by other routes. A preferred dosage is ˜1 μg/kg to ˜1 mg/kg, with ˜5 μg/kg to ˜200 μg/kg particularly preferred. It will be evident to those skilled in the art that the number and frequency of administration will be dependent upon the response of the host. “Pharmaceutically acceptable carriers” for therapeutic use are well known in the pharmaceutical art, and are described, for example, in Remington's Pharmaceutical Sciences, Mack Publishing Co. (A.R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used. “Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention. The pharmaceutical compositions that contain one or more salicylic acid binding proteins may be in any form that allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal. The term parenteral as used herein includes subcutaneous injections, intravenous, intramuscular, intrastemal, intracavemous, intrathecal, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection/as two examples. When intended for oral administration, preferred compositions contain, in addition to one or more SARP molecules one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile. It may also be desirable to include other components in the preparation, such as delivery vehicles including but not limited to aluminum salts, water-in-oil emulsions, biodegradable oil vehicles, oil-in-water emulsions, biodegradable microcapsules, and liposomes.

While any suitable carrier known to those of ordinary skill in the art may be employed in the pharmaceutical compositions of this invention, the type of carrier will vary depending on the mode of administration and whether a sustained release is desired. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. In this regard, it is preferable that the microsphere be larger than approximately 25 μm.

Pharmaceutical compositions may also contain diluents such as buffers, antioxidants such as ascorbic acid, low molecular weight (less than ˜10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Preferably, product is formulated as a lyophilizate using appropriate excipient solutions (e.g., sucrose) as diluents.

A liquid composition intended for either parenteral or oral administration should contain an amount of SARP/or other therapeutic agent identified according to methods herein described such that a suitable dosage will be obtained. Typically, this amount is at least 0.01 wt% of a SARP and/or delivered therapeutic agent in the composition. Preferred compositions and preparations are prepared so that a parenteral dosage unit contains between 0.01 to 1% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Exemplary topical formulations may contain a concentration of SARP of from ˜0.1 to ˜10% w/v (weight per unit volume). The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. In the methods of the invention, the pharmaceutical compositions comprising SARP of the present invention, and/or comprising one or more agents that alter at least one salicylate biological effect as described herein, may be administered through use of insert(s), bead(s), timed-release formulation(s), patch(es) or fast-release formulation(s).

### Others

The present invention contemplates one or more expression vectors that individually or collectively express any one or more of the SARPs described herein. For example, in one emboidment, an expression vector encoding an SARP having an ion protein at m/z at any one or more of the following: m/z 854, 900, 924, 1178, 1220, using an ESI-MS spectra.

The present invention also contemplates a method for detecting a metabolic condition in a subject which includes assaying for a confomration state of an SARP in a sample from the subject. For example, a brain tissue sample can be assayed to determine state of an SARP in the sample, e.g., using purificiation techniques as described herein, followed by mass-spec analaysis. If the SARP is detected in the A-state then a report is provided to the subject indicating that the subject is afflicted with a metabolic disorder such as Alzhemer's, cancer, diabetes, Reye's syndrome, etc. If the SARP is detected in the B-state, then a report is provided to the subject indicating that the subject is not afflicte d with such a condition. In another embodiment, a sample from the subject is contacted with antibodies directed to SARP (state A and/or state B). The presence of state A is indicative of a condition as described below and the subject is provided a report indicating the same. The presence of state B is indicative of the lack of such condition.

In one embodiment, the present disclosure contemplates a kit with antibodies, such that a first antibody selectively binds SARP state A and a second antibody selectively binds SARP in state B.

In one embodiment, the present disclosure contemplates a drug delivery complex comprising SARP coupled to a therapeutic agent wherein the therapeutic agent is released from the complex when the SARP is in its state B and wherein the therapeutic agent is maintained in the complex when the SARP is in its state A.

In one embodiment, the disclosure contemplates a method for treating a metabolic condition in an organism comprising administering to the subject an agent that maintains SARP in a single state for a therapeutic period of time. Such agent can be a small molecule, antibody, protein or peptide that selectively binds SARP or modulates its activity. In some instances, the agent maintains SARP in its B state (such as when the organism is a human). In some instances the agent maintains SARP in its A state (such as when the organism is a plant). In some instances the metabolic condition is aging, obesity, Alzheimer's, or cancer.

The following Examples are offered by way of illustration and not limitation.

### EXAMPLES

### Example 1

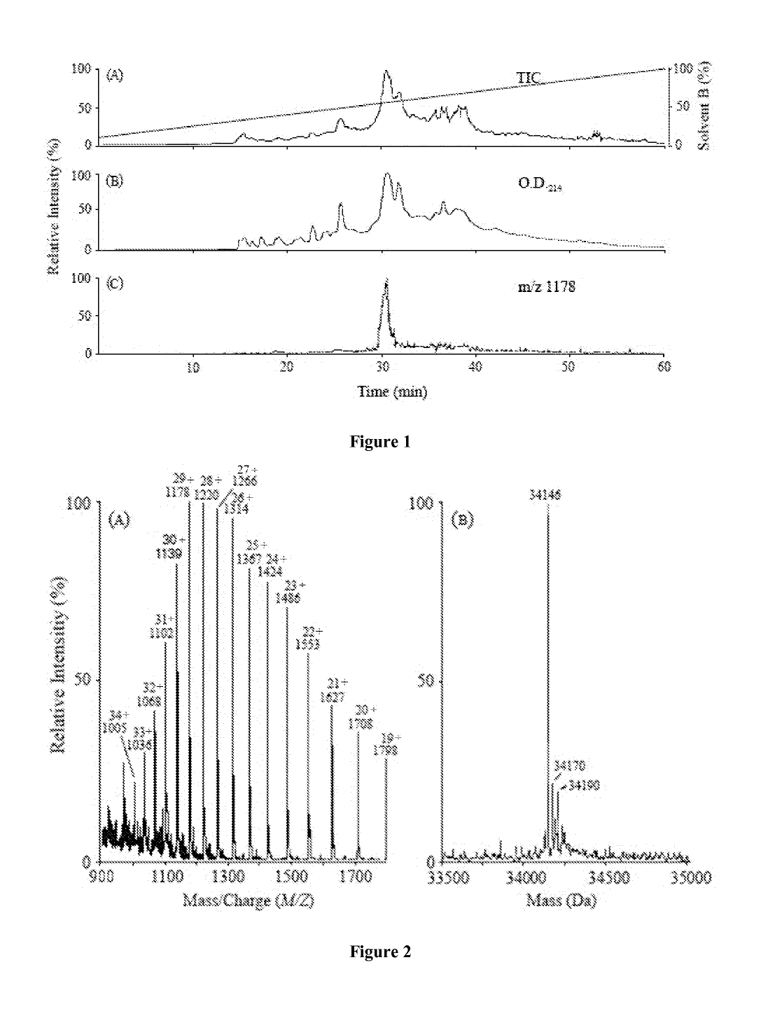

FIG. 1 illustrates purification of SARP from thermogenic Sauromatum appendix using perfusion chromatography, RP-HPLC-ESI-MS. TIC (total ion current) chromatogram with solvent gradient overlay (A); absorbance at 214 nm (B); ion current chromatogram of m/z 1178, corresponding to [M+29H]²⁹⁺ CSD of the protein (C). Solvent A is 0.05% TFA in aqueous solution and solvent B is 0.05% aqueous TFA in ACN:IP at a ratio 2:1.

TABLE I
Solubility of SARP in isopropanol solutions with different
water contents. D-day appendix tissue was extracted with
the isopropanol buffer. The amount of SARP separated
on the RP-HPLC/MS-ESI was determined using the
MassLynx software. Each extraction was done with
50 g of Sauromatum appendix.

| Isopropanol (%) | SARP (ng/g fresh wt) |
|---|---|
| 50 | 560 |
| 75 | 80 |
| 95 | 30 |

TABLE II
The abundance of SARP at different developmental
stages of the Sauromatum appendix. The amount
SARP separated on the RP-HPLC/MS-ESI was determined
using the MassLynx software. The protein was extracted
from 15-20 g of appendix at different developmental stages.
During anthesis, the level of SARP increased 17 fold
from D-3 to D-2 and an additional increase was observed
on D-day, the day of heat-production. The next day, the
level dropped to an undetectable level.

| Stage of Development | SARP (ng/g fresh wt.) |
|---|---|
| D-4 | 3 |
| D-3 | 3 |
| D-2 | 50 |
| D-1 | 170 |
| D-day | 350 |
| D + 1 | Undetectable |

TABLE III
Sub-cellular distribution of SARP. Twenty three g of a
D-day appendix was ground in the mitochondria isolation
buffer and the presence of the protein in different fractions
was determined. SARP from the Sauromatum appendix
was present in the soluble part of the cytoplasm and was
also located in different organelles such as the mitochondria
and possibly in nuclei. The mitochondrial preparation contained
1.4 mg of protein.

| Fraction | SARP (ng/g fresh wt.) |
|---|---|
| 1,000 g pellet | 12 |
| 5,000 g pellet | 10 |
| 27,000 g pellet | 17 |
| Cell membranes, 400,000 g pellet | Undetectable |
| Supernatant of 400,00 g pellet | 25 |

TABLE IV
The presence of SARP in thermogenic organs of different plants.
D-day appendices of Arum italicum (40 g) and Amorphophallus
konjac (100 g). Dracunculus vulgaris appendix 2 to 1 days
before heat-production (44.5 g). Appendages of Victoria flowers
on the second day of heat-production (68 g), and sporophylls of
male cones of Encephalartos ferox during the week of
heat-production (40 g). SARP was found in thermogenic floral
organs of members of three plant families: Araceae (A. italicum,
A. konjac, D. vulgaris), Nymphaeceae (V. cruziana),
and Cycadaceae (E. ferox), suggesting that SARP is responsible
for heat production in all thermogenic plants.

| Plant Species | SARP (ng/g fresh wt.) |
|---|---|
| A. Italicum | 1 |
| A. konjac | 20 |
| D. vulgaris | 70 |
| V. cruziana | 9 |
| E. ferox | 7 |

TABLE V
The effect of SA on the levels of SARP in the Sauromatum
appendix. D-1 appendices were cut off the inflorescence 2 h
after onset of daylight and placed in 10 μM SA or in water
as a control. The next morning when heat was produced the
protein was extracted and separated on the RP-HPLC column
and its amount was determined. When the Sauromatum
appendix was cut off from the inflorescence, the amount of
SARP decreased. Application of SA to a section of the
appendix further decreased the amount of SARP.

| D-1 Appendix | SARP (ng/g fresh wt.) |
|---|---|
| Control | 160 |
| SA-treated | 30 |

### Example 2

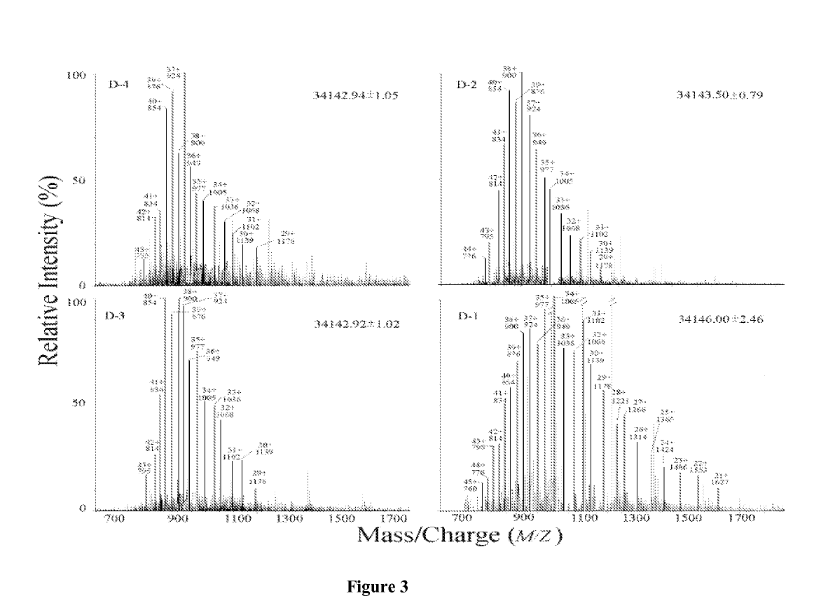

FIG. 3 illustrates conformational changes in SARP as purified according to change from state A towards state B of SARP using ESI-MS.

On D-1 a shift to a higher m/z values followed a strong declined in the lower m/z values. The change to a lower m/z values was a result of inaccessibility of protonable sites buried inside a more compact form of cytosolic SARP, henceforth referred as state B. State B must have a similar cross sectional area to the expanded conformation, henceforth referred as state A, as the additional charge state ions 34+ to 28+ are in-line with the 37+ and 38+ charge state ions. On D-1, the broadness of the CSD reflects the different conformations two states, states A and B with overlapping CSD. Whereas from D-4 until D-1 state A was the predominate state. On D-day state B was the predominate state. It is unlikely that the asymmetric CSD could be induced by the electrospray processes asymmetric partitioning that occurs in the gas phase because carbonic anhydrase, our calibration standard, yielded the correct deconvolution information under the same running conditions. It strongly suggests that state B of SARP is involved in thermogenicity.

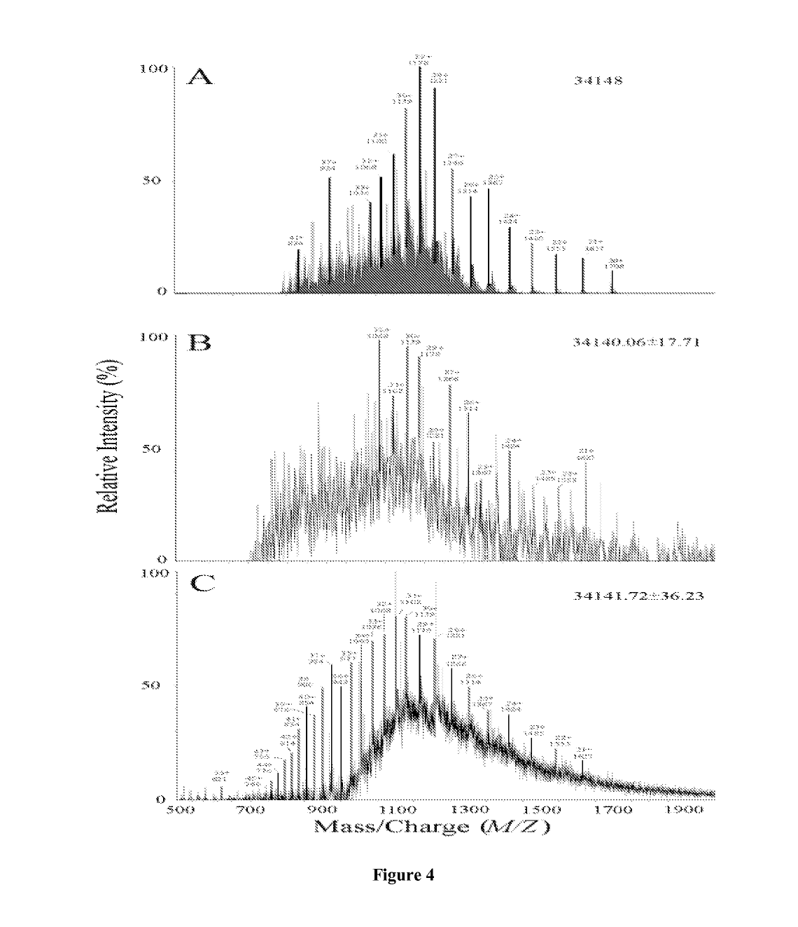

Under denaturing conditions in which cytosolic SARP was incubated for 2 h in 6 M GuHCl, the magnitude of m/z 1102 corresponding to [M+31H]³¹⁺ was maximum (FIG. 4C). SARP electrosprayed from denaturing conditions acquired significantly higher multiply charged ion envelope (lower m/z value) as expected (state A) but SARP was only partially denatured because high m/z values (state B) were still detectable.

### Example 3

SARP in aqueous solutions, pH 7.0 can self-assembled into well-defined morphologies. The morphologies were divided into five different assembly pathways. For such self-assembly conformational switches must be built in SARP molecules. When SARP binds at one site to another SARP molecule it may cause a conformational change in another site resulting in sequential conformational changes followed by assembly steps. In order of SARP to adopt several conformations under physiological condition it is has to be in the expanded state. SARP may be wobbling between different conformational states in solution and once a group of SARP molecules has formed a stable seed or nucleus, other molecules join in and acquire the same structure. SARP molecules that are less stable as monomers may have a greater chance of assuming alternative native states as multimers. SARP must possess a fine balance of attractive and repulsive forces that result in rich morphologies, and it must have distinct hydrophilic and hydrophobic parts. The linear polymerization could be formed by intra-molecular interactions of head-to-tail interaction whereas circular polymerization could be formed by intermolecular interaction of SARP molecule with itself The changes in morphology from spherical to vesicle-like and rod-like assembly, and to tree branches ensembles may involve conformational changes.

### Example 4

RL is in an oligometric form in water and that RL has a high water content. The swelling of a polymer gel is usually determined by the interaction between the oligomer molecules to form a network and its affinity for water.²⁶ Water molecules prevent the collapse of the network and the network entraps the water creating a microenvironment different from the surrounding environment. There are several proteins that can form hydrogel, including NCK (non-catalytic region of tyrosine kinase)/WASP (Wiskott-Aldrich syndrome protein) family proteins and nephrin, which, are involved in actin signaling (Tompa Intrins Prot Disorder 2013, 1:e24068; Li, Nature 2012, 483:336). Artificial proteins have also been used to form hydrogels (Petka et al., 1998, Science 281:389; Dinerman et al., Biomaterials 2002, 23:4203).

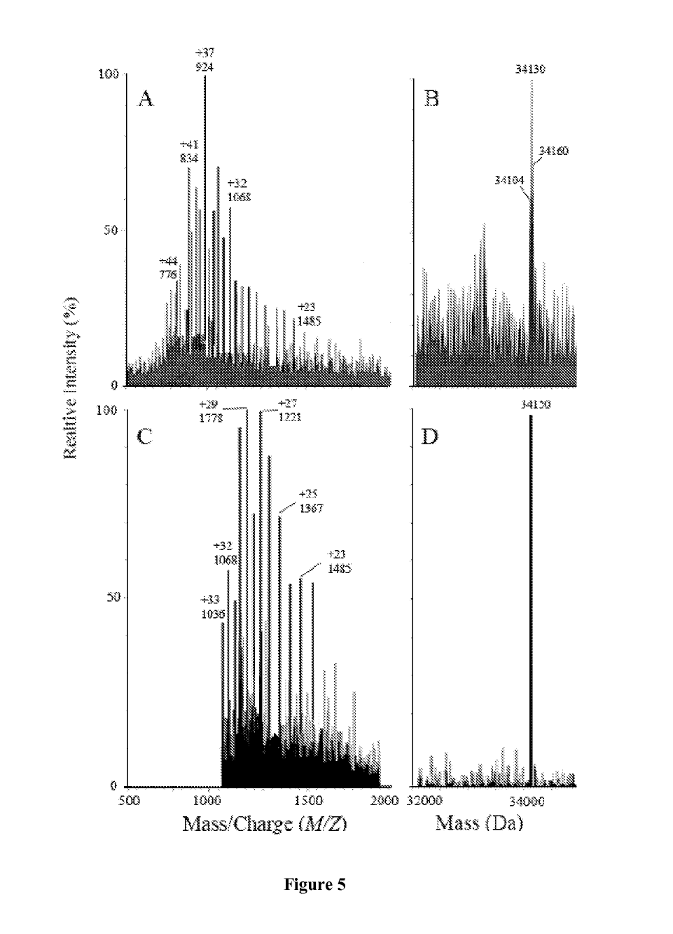

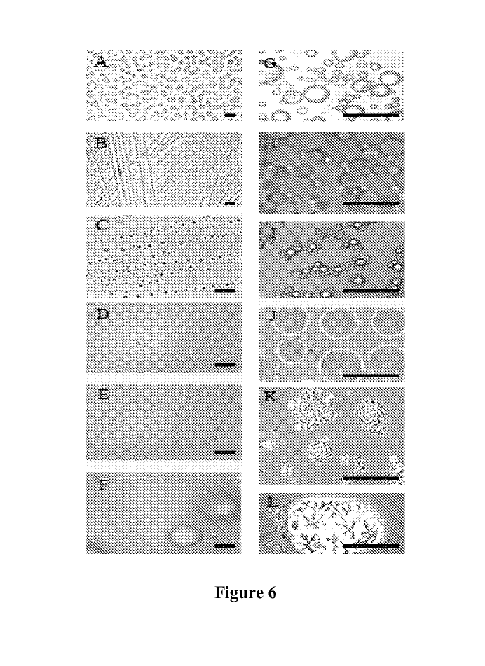

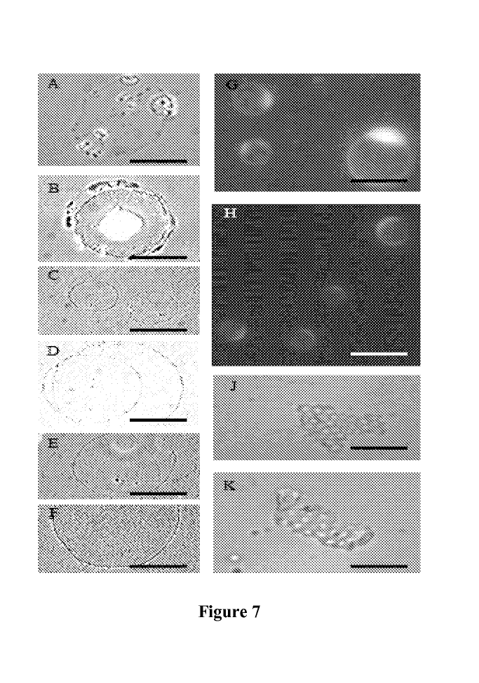

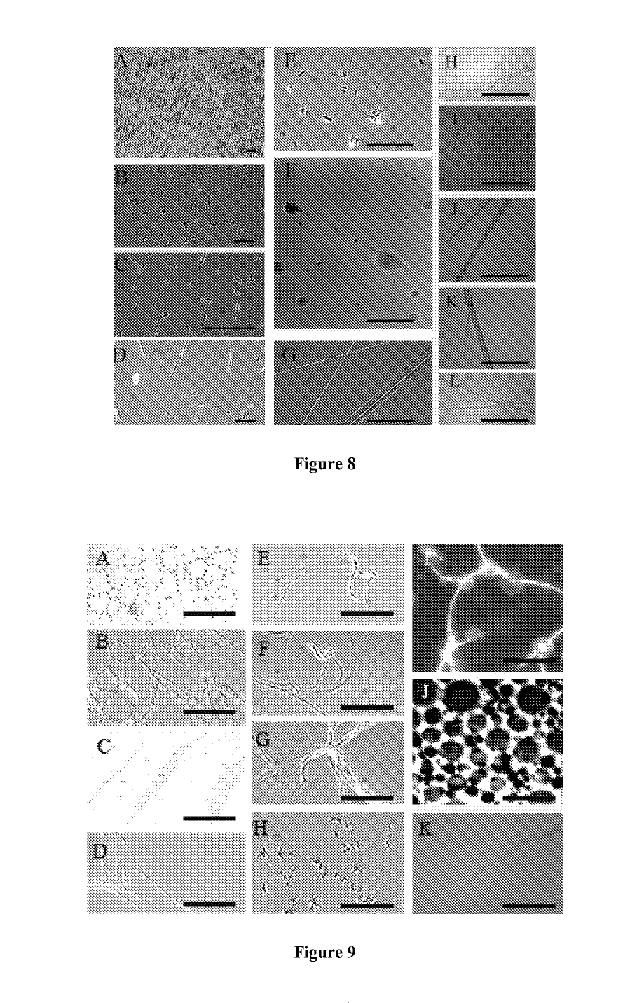

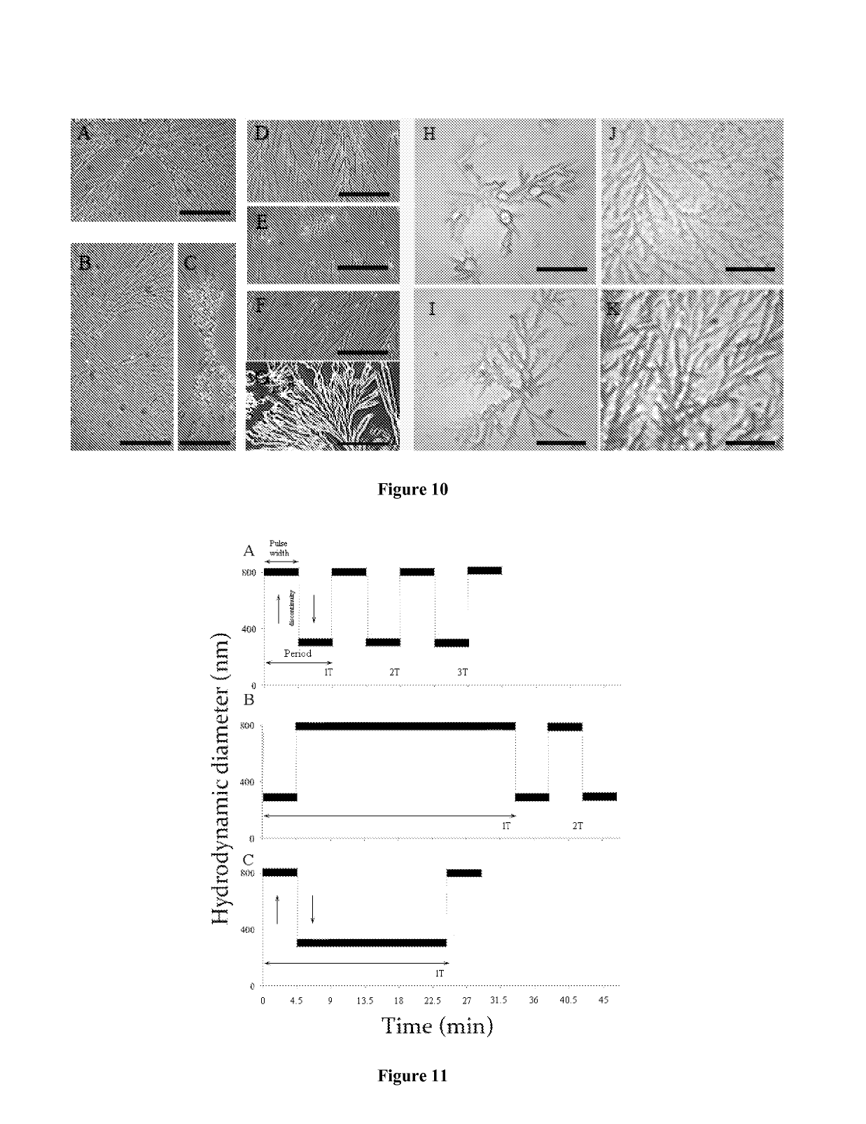

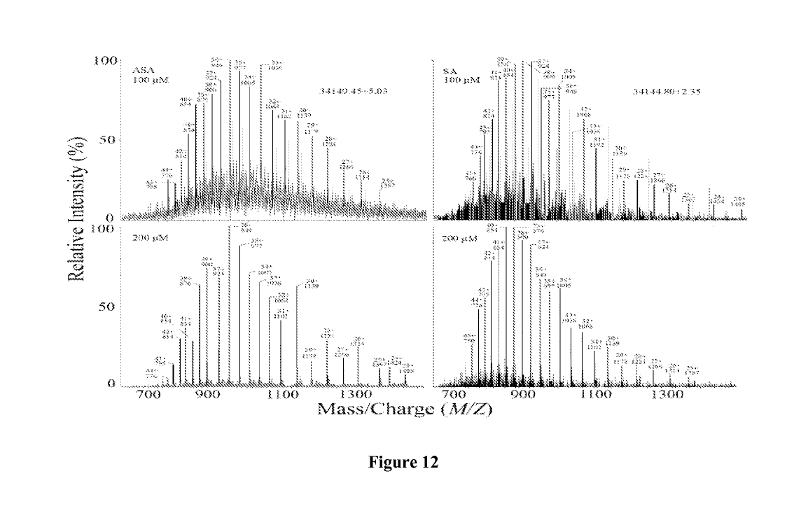

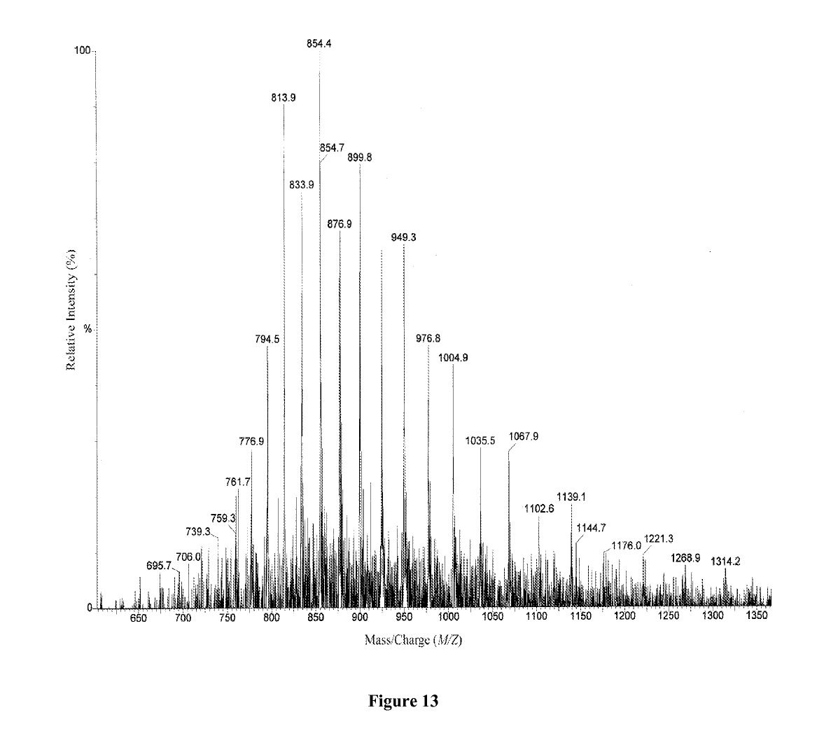

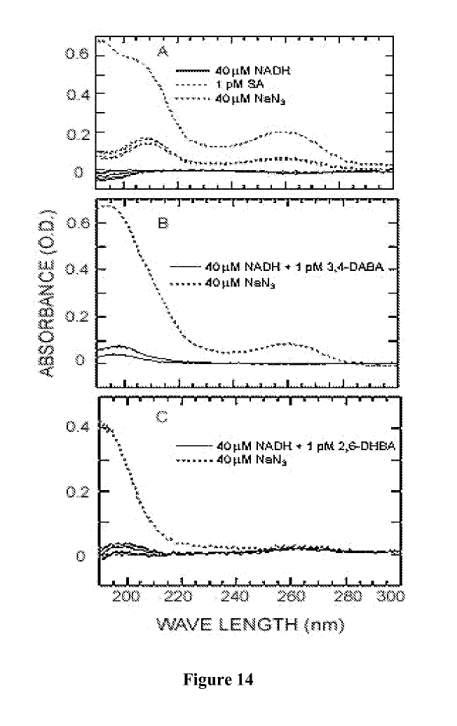

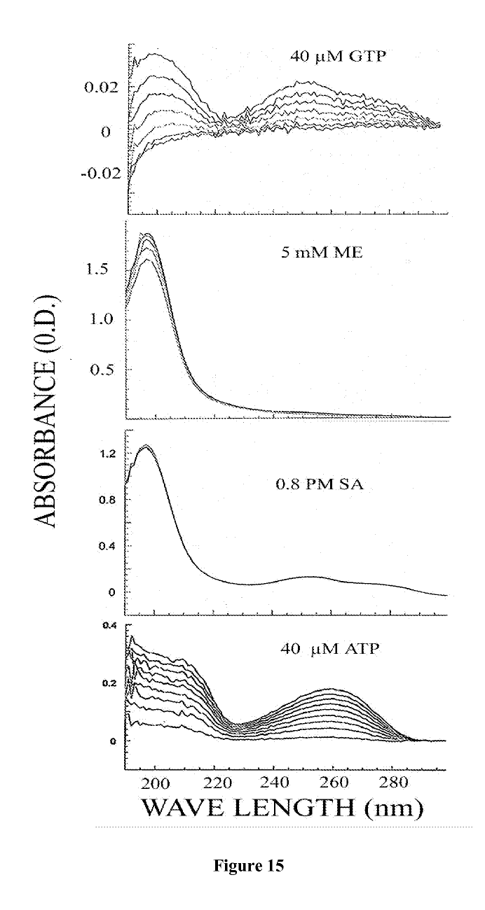

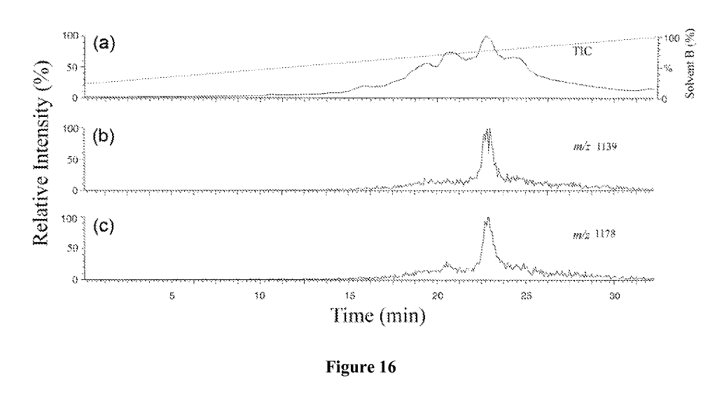

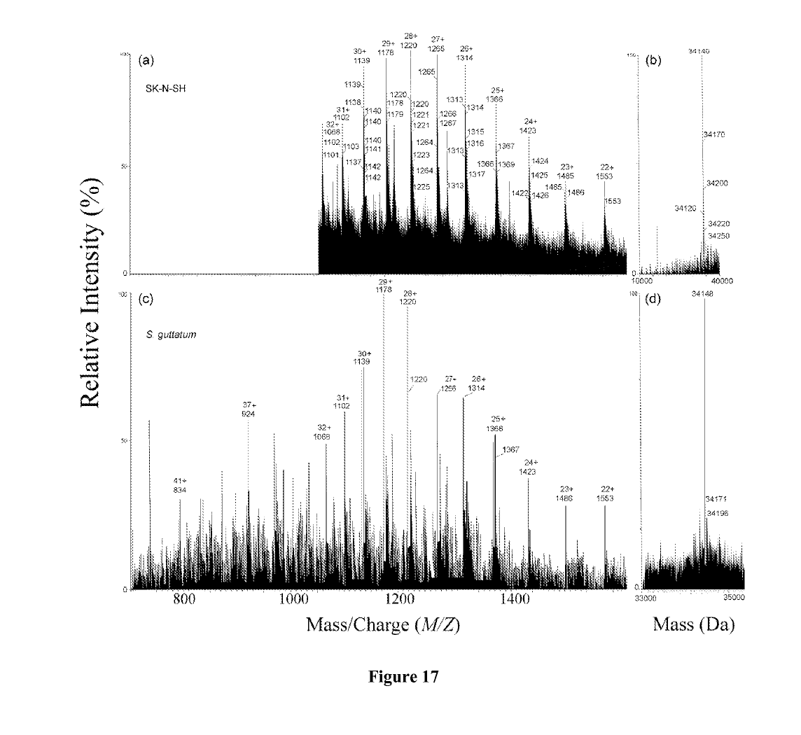

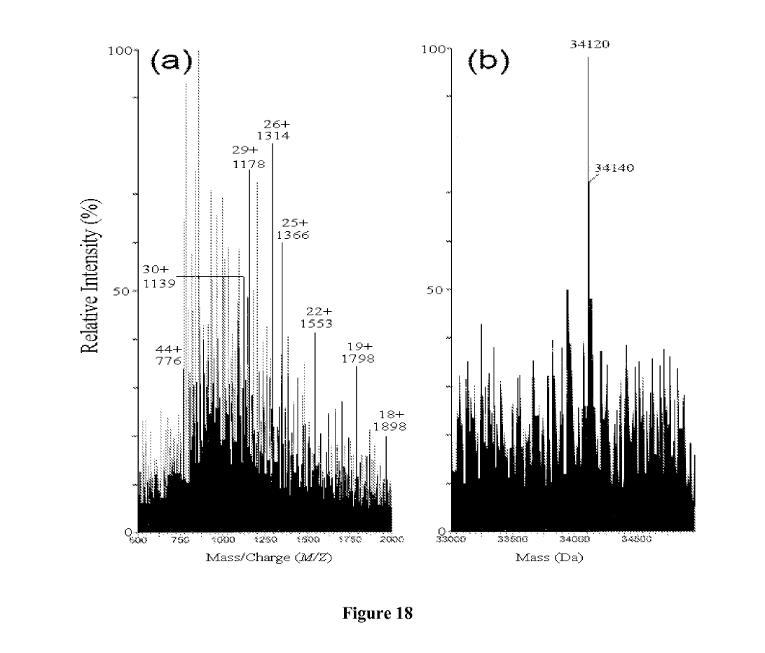

Laser dynamic light scattering (DLS) measurements of SARP in water demonstrated the unusual physical ability SARP to condense and relax. Upon addition of water to the dry protein at a final concentration of 0.1 pM, micelles formed with a uniform size of ˜800 nm (FIG. 5). Upon addition of DTT or β-mercaptoethanol the micelle size reduced to ˜300 nm, and after 20 min, the size increased back to ˜800 nm. Addition of 1 pM SA, at a molar ratio [SARP]/[SA]<0.1, immediately decreased the micelle size. SARP molecules became intertwined and formed a gel that expanded and shrank in an oscillatory manner, every 4-5 min. Addition of 10 μM ATP stopped the oscillation for ˜30 min. and then it resumed with the same kinetics.

### Example 5

Monomeric SARP undergoes a large-scale conformation transition from one state to another in the Sauromatum appendix during development. SARP envelope shifted from [M+37H]⁺³⁷ and [M+39H]⁺³⁹ (state A) to [M+29H]⁺²⁹ (state B). Application of 10 μM SA to pre D-day appendices induced a transition from state A in which SA undetectable in the tissue to state B in which SA concentration high in the tissue. The concentration of SA in the appendix tissue is ˜1 μg/g fresh wt⁴, and it is equivalent to ˜7 μM, within the range of this experiment. At higher concentrations of SA and 2,6-DHBA (100 and 200 μM) SARP adopted conformations that are more extended than state A. It strongly suggests that the conformation of SARP can switch between extended and compact conformations depending on the inducer concentration.

### Example 6

TABLE VI
The reduction of NBT, MTT and NeoT by SARP in the presence
of pyridine. NBT, MTT and NeoT at a final concentration of
4 μM and pyridine was added to the reaction mixture at a
final concentration of 30%. After 5 min of incubation at room
temperature, the samples were frozen. Later, the absorbance
of the control and samples with tetrazolium salts was determined.
The data are a mean of two determinations. Three tetrazolium
salts that can accept electrons were used to determine whether
SARP can transfer electrons under aerobic and anaerobic conditions.
When the formation of formazan was monitored at 530 nm, no
significant reduction of NBT was detected. Under anaerobic
conditions, NBT, NeoT and MTT were reduced by SARP.

| Tetrazolium salt (μM) | O.D.₅₃₀ |
|---|---|
| NBT | 0.14 |
| MTT | 0.17 |
| NeoT | 0.06 |

### Example 7

SARP tryptic peptide sequencing was done by LC/ESI-MS/MS using an ion trap mass spectrometer, and searches of public databases for tryptic digest similarities were performed. Four hundred ng of SARP was digested with trypsin and alkylated. The Blast search provided one peptide that exhibited a 100% match, namely of SARP-derived tryptic peptide FLPSEFGNDVDR [SEQ ID NO:1] with a fragment of a plant reductase, phenylcoumaran benzylic ether reductase from Arabidopsis thaliana, gi 8778426 [SEQ ID NO:6] and Tsuga heterophlla; gi 7578907 [SEQ ID NO:5] and gi7578909 [SEQ ID NO:4]. A positive match of SEQ ID NO:1 was also found in reductases from other plants and a a positive match to SEQ ID NO:2 was found in other organisms.

The enzyme phenylcoumaran benzylic ether reductase is related to other plant reductases, isoflavone reductase (Oommen et al., 1994 Plant Cell 6:1789) and pinoresinol-lariciresinol reductase. (Dinkova-Kostova et al., 1996 J. Chem. Biol. 271:29473; Gang et al., 1999 J. Biol. Chem. 274:7516). All three plant reductase classes are involved in the biosynthesis of related phenylpropanoid-derived plant defense compounds. The plant reductase enzyme isoflavone reductase in Arabidopsis also includes this short peptide sequence; plants contain a large number of proteins homologous to isoflavone reductase from legumes (Shoji et al., 2002 Plant Mol. Biol. 50:427; Schmitz et al., 1999 J. Allerg. Clin. Immunol. 104:991). Thus, phenylcoumaran benzylic ether reductase may be a homolog of isoflavone reductase with a similar enzymatic activity, or it may be a “moonlighting” protein, i.e., a protein with more than one function (Jeffrey, 1999 Trends Biochem. Sci. 24:8). Compositions and methods relating to plant reductases, including polynucleotide sequences encoding several plant reductases and the encoded polypeptide sequences, are also disclosed in U.S. Pat. Nos. 6,210,942 and 5,750,399 and in Patent Application Publication No. 2002/0177527 A1; as herein described, certain embodiments of the present invention therefore contemplate the use as a SARP of any one or more of these and other plant reductases or portions of such reductases (e.g., fragments that are capable of mediating a salicylate biological effect according to the instant disclosure), in particular, where the plant reductase comprises SEQ ID NO:1 or 2 or a homologous sequence comprising 1, 2, 3, 4, 5 or 6 amino acid substitutions, insertions or deletions relative to SEQ ID NOS:1 or 2, the substitutions preferably being “conservative” substitutions as known in the art.

Based on the rare partial sequence match between SARP and a portion of the above described reductases, and in particular the Arabidopsis isoflavone reductase SEQ ID NO:6, the invention contemplates a family of plant-derived SARP that are in fact reductases which comprise the peptide sequence set forth in SEQ ID NO:1 or 2 (or a homologous sequence) and which exhibit SA sensitivity, UV absorbance spectra and other properties as described herein for SARP, but that need not be limited to the 34.1 kDa Sauromaium SARP. Hence, certain embodiments of the invention relate to methods for identifying an agent that alters a biological effect as described above, but that may use any of the herein described plant reductases (including those disclosed in the cited publications) in place of the 34.1 kDa Sauromatum SARP. Other examples of related proteins include those shown in Table VIII.

TABLE VII
Plant Reductases and Homologues Thereof

| | | |
|---|---|---|
| 1 | FLPSEFGNDVDR | |
| 2 | FLPSEF | |
| 3 | gi\|15223574\|gb\|NP_173385.1 | NmrA-like negative . . . |
| 4 | gi\|7578909\|gb\|AAF64181.1\| | phenylcoumaran benzylic ether red . . . |
| 5 | gi\|7578907\|gb\|AAF64180.1\| | phenylcoumaran benzylic ether red . . . |
| 6 | gi\|8778426\|gb\|AAF79434.1\| | F18O14.30 [Arabidopsis thaliana] |
| 7 | gi\|7578905\|gb\|AAF64179.1\| | phenylcoumaran benzylic ether red . . . |
| 8 | gi\|7578901\|gb\|AAF64177.1\| | phenylcoumaran benzylic ether red |
| 9 | gi\|29466466\|emb\|CAD79341.1\| | phenylcoumaran benzylic ether red . . . |
| 10 | gi\|7578899\|gb\|AAF64176.1\| | leucoanthocyanidin reductase [D . . . |
| 11 | gi\|1708422\|sp\|P52578\|IFRH_SOLTU | phenylcoumaran benzylic ether red . . . |
| 12 | gi\|19310585\|gb\|AAL85023.1\| | Isoflavone reductase homolo . . . |
| 13 | gi\|10764491\|gb\|AAG22740.1\| | putative NAD(P)H oxidoreductase, . . . |
| 14 | gi\|3243234\|gb\|AAC24001.1\| | allergenic isoflavone reductase- . . . |
| 15 | gi\|4731376\|gb\|AAC05116.2\| | isoflavone reductase related prot . . . |
| 16 | gi\|7484603\|pir\|\|T08106 | isoflavone reductase homolog Bet . . . |
| 17 | gi\|15234993\|ref\|NP_195634.1\| | 2′-hydroxyisoflavone reductase (EC 1 . . . |
| 18 | gi\|6573171\|gb\|AAF17578.1\| | isoflavone reductase, putative . . . |
| 19 | gi\|18410820\|ref\|NP_565107.1\| | isoflavone reductase homolog 2 [G . . . |
| 20 | gi\|25344798\|pir\|\|C96783 | isoflavone reductase, putative . . . |
| 21 | gi\|19847822\|gb\|AAK27264.1\| | probable NADPH oxidoreductase, 1409 . . . |
| 22 | gi\|3415126\|gb\|AAC32591.1\| | isoflavone reductase-like protei . . . |
| 23 | gi\|38492949\|pdb\|1QYC\|A | phenylcoumaran benzylic ether red- |
| 24 | gi\|6525021\|gb\|AAF15291.1\| | Chain A, Crystal Structures Of Pinor . . . |
| 25 | gi\|7578897\|gb\|AAF64175.1\| | isoflavone reductase-like NAD(P)H . . . |
| 26 | gi\|7578895\|gb\|AAF64174.1\| | phenylcoumaran benzylic ether red . . . |
| 27 | gi\|7578911\|gb\|AAF64182.1\| | phenylcoumaran benzylic ether red . . . |
| 28 | gi\|1708423\|sp\|P52579\|\|FRH_TOBAC | phenylcoumaran benzylic ether red . . . |
| 29 | gi\|6573169\|gb\|AAF17577.1\| | Isoflavone reductase homolo . . . |
| 30 | gi\|1708427\|sp\|P52576\|IFR_PEA | isoflavone reductase homolog 1 [G . . . |
| 31 | gi\|11127951\|gb\|AAG31154.1\| | Isoflavone reductase (IFR) (NA . . . |
| 32 | gi\|9255858\|gb\|AAF86332.1\| | isoflavone reductase [Lotus com . . . |
| 33 | gi\|15222190\|ref\|NP_177664.1\| | isoflavone reductase [Medicago tr . . . |
| 34 | gi\|1708426\|sp\|P52575\|IFR_MEDSA | isoflavone reductase, putative . . . |
| 35 | gi\|99982\|pir\|\|S17744 | isoflavone reductase (IFR) ( . . . |
| 36 | gi\|1708421\|sp\|P52580\|IFRH_MAIZE | 2′-hydroxyisoflavone reductase (EC 1.3 . . . |
| 37 | gi\|15222191\|ref\|NP_177665.1\| | Isoflavone reductase homolo . . . |
| 38 | gi\|7488639\|pir\|\|T07095 | isoflavone reductase, putative . . . |
| 39 | gi\|1708425\|sp\|Q00016\|IFR_CICAR | 2′-hydroxyisoflavone reductase (EC 1 . . . |
| 40 | gi\|32413947\|ref\|XP_327453.1\| | Isoflavone reductase (IFR) ( . . . |
| 41 | gi\|3114901\|emb\|CAA06707.1\| | hypothetical protein [Neurospo . . . |
| 42 | gi\|3114899\|emb\|CAA06706.1\| | phenylcoumaran benzylic ether re . . . |
| 43 | gi\|3114903\|emb\|CAA06708.1\| | phenylcoumaran benzylic ether re . . . |
| 44 | gi\|18250364\|gb\|AAL61542.1\| | phenylcoumaran benzylic ether re . . . |
| 45 | gi\|34894098\|ref\|NP_908374.1\| | isoflavone reductase-like protei . . . |
| 46 | gi\|7578917\|gb\|AAF64185.1\| | putative isoflavone reductase . . . |
| 47 | gi\|6906849\|gb\|AAF31166.1\| | Pinoresinol-lariciresinol reducta . . . |
| 48 | gi\|7542585\|gb\|AAF63509.1\| | Pinoresinol-lariciresinol reducta . . . |
| 49 | gi\|38492951\|pdb\|1QYD\|A | pinoresinol-lariciresinol reducta . . . |
| 50 | gi\|1352187\|sp\|P48420\|CP78_MAIZE | Chain A, Crystal Structures Of Pinor . . . |
| 51 | gi\|24653657\|ref\|NP_725398.1\| | Cytochrome P450 78A1 (CYPLX . . . |
| 52 | gi\|15222571\|ref\|NP_174490.1\| | Additional sex combs CG8787-PA . . . |
| 53 | gi\|34866316\|ref\|XP_232674.2\| | pinoresinol-lariciresinol redu . . . |
| 54 | gi\|15236330\|ref\|NP_193102.1\| | similar to hypothetical protei . . . |
| 55 | gi\|21592830\|gb\|AAM64780.1\| | pinoresinol-lariciresinol redu . . . |
| 56 | gi!33636671\|gb\|AAQ23633.1\| | pinoresinol-lariciresinol reduct . . . |
| 57 | gi\|26351059\|dbj\|BAC39166.1\| | AT27514p [Drosophila melanogaster] |
| 58 | gi\|27734849\|ref\|NP_775790.1\| | unnamed protein product [Mus mu . . . |
| 59 | gi\|12855458\|dbj\|BAB30342.1\| | hypothetical protein MGC34646 . . . |
| 60 | gi\|1708424\|sp\|P52581\|IFRI_LUPAL | unnamed protein product [Mus mu . . . |
| 61 | gi\|7578915\|gb\|AAF64184.1\| | Isoflavone reductase homolo . . . |
| 62 | gi\|1769556\|gb\|AAC49608.1\| | pinoresinol-lariciresinol reducta . . . |
| 63 | gi\|7512029\|pir\|\|T13748 sex comb protein | Forsythia x intermedia (+)-pinore . . . |
| 64 | gi\|24649775\|ref\|NP_651283.1\| | fruit fly (Drosop . . . |
| 65 | gi\|33589456\|gb\|AAQ22495.1\| | CG6879-PA [Drosophila melanoga . . . |
| 66 | gi\|28901209\|ref\|NP_800864.1\| | RE05911p [Drosophila melanogaster] |
| 67 | gi\|21674763\|ref\|NP_662828.1\| | putative type III secretion sy . . . |
| 68 | gi\|16081866\|ref\|NP_394269.1\| | 8-amino-7-oxononanoate synthas . . . |
| 69 | gi\|38103262\|gb\|EAA49979.1\| | indole-3-glycerol-phosphate sy . . . |
| 70 | gi\|32265805\|ref\|NP_859837.1\| | hypothetical protein MG10688.4 [ . . . |
| 71 | gi\|29841241\|gb\|AAP06273.1\| | conserved hypothetical protein . . . |
| 72 | gi\|16752894\|ref\|NP_445165.1\| | hypothetical protein with Src ho . . . |
| 73 | gi\|23110320\|ref\|ZP_00096478.1\| | conserved hypothetical protein . . . |
| 74 | gi\|23007479\|ref\|ZP_00049332.1\| | COG1629: Outer membrane rece . . . |
| 75 | gi\|29348095\|ref\|NP_811598.1\| | COG0018: Arginyl-tRNA synthe . . . |
| 76 | gi\|32041024\|ref\|ZP_00138607.1\| | putative cation efflux transpo . . . |
| 77 | gi\|15618074\|ref\|NP_224358.1\| | hypothetical protein [Pseudo . . . |
| 78 | gi\|15835685\|ref\|NP_300209.1\| | CT147 hypothetical protein [Ch . . . |
| 79 | gi\|17542960\|ref\|NP_501614.1\| | CT147 hypothetical protein [Ch . . . |
| 80 | gi\|32475155\|ref\|NP_868149.1\| | putative protein family member . . . |
| 81 | gi\|15596222\|refJNP_249716.1\| | serine/threonine kinase Pkn10 . . . |
| 82 | gi\|38099423\|gb\|EAA46772.1\| | probable porin [Pseudomonasae . . . |
| 83 | gi\|23507829\|ref\|NP_700499.1\| | hypothetical protein MG10466.4 [ . . . |
| 84 | gi\|23056305\|ref\|ZP_00082347.1\| | PF70 protein [Plasmodium falci . . . |
| 85 | gi\|29246665\|gb\|EAA38253.1\| | COG1109: Phosphomannomutase . . . |
| 86 | gi\|21672089\|gb\|AAM74451.1\| | GLP_15_6000_10586 [Giardia Iambi . . . |
| 87 | gi\|6679429\|ref\|NP_032928.1\| | Putative retroelement [Oryza sat . . . |
| 88 | gi\|23014322\|ref\|ZP_00054144.1\| | placenta! protein 11 related [M . . . |
| 89 | gi\|34894598\|ref\|NP_908624.1\| | COG0018: Arginyl-tRNA synthe . . . |
| 90 | gi\|37533764\|ref\|NP_921184.1\| | putative sarcosine oxidase [Or . . . |
| 91 | gi\|37533772\|refJNP_921188.1\| | putative retroelement [Oryza s . . . |
| 92 | gi\|15219183\|ref1NP_173077.1\| | putative retroelement [Oryza s . . . |
| 93 | gi\|21672085\|gb\|AAM74447.1\| | protein kinase family [Arabido . . . |
| 94 | gi\|23041158\|ref\|ZP_00072635.1\| | Putative retroelement [Oryza sat . . . |
| 95 | gi\|38346483\|emb\|CAE03722.2\| | COG1649: Uncharacterized j >ro . . . |
| 96 | gi\|19703371\|ref\|NP_602933.1\| | OSJNBa0021F22.16 [Oryza sativa . . . |
| 97 | gi\|31209293\|ref\|XP_313613.1\| | Transcription-repair coupling . . . |
| 98 | gi\|15672909\|ref\|NP_267083.1\| | ENSANGP00000003616 [Anopheles . . . |
| 99 | gi\|29248627\|gb\|EAA40156.1\| | teichoic acid biosynthesis pro . . . |
| 100 | gi\|38374311\|gb\|AAR19360.1\| | GLP_393_17539_16058 [Giardia lam . . . |
| 101 | gi\|21757260\|dbj\|BAC05072.1\| | core protein [Hepatitis B virus] |
| 102 | FLPS | |

What is claimed is:

1. An isolated and purified salicylic acid receptor protein (SARP) that binds salicylic acid at a concentration less than 10⁻⁹ molar (M)

2. The SARP of claim 1 wherein said SARP binds salicylic acid at a concentration less than 10⁻¹⁰, 10⁻¹¹ or 10⁻¹² _{molar (M)}

3. An isolated and purified SARP that binds salicylic acid wherein said SARP has an in vitro volume phase transition.

4. The SARP of claim 3 wherein the volume phase transition occurs at least once every 30 minutes.

5. The SARP of claim 3 wherein the volume phase transition occurs at least once every 5 minutes.

6. The SARP of claim 3 wherein the SARP in water, according to DLS, results in uniform size of ˜800 nm and ˜300 nm alternating at repeating intervals.

7. An isolated and purified SARP having at least two conformational states.

8. An isolated and purified SARP having an ESI-MS spectra with maxima at one or more of the following: m/z 854, 900, 924, 1178, 1220.

9. An antibody or antibody fragment that selectively binds the SARP of any of the above claims.

10. A method for identifying agents that modulate SARP comprising performing a competition assay between a first agent selected from the group consisting of SA, ASA, and 2,6-DHBA and a second agent and their binding affinity to an SARP, and selecting the second agent that outcompetes binding of the first agent to the SARP for drug development.

11. A method for assaying for a metabolic condition in a subject comprising: assaying for the presence of a state A or state B conformation of a SARP in a sample from the subject and providing a report indicating if the subject is susceptible to or is experiencing such metabolic condition.

12. An isolated and purified salicylic acid receptor protein (SARP) having at least 50%, 60%, 70%, 80%, 90%, 95%, 99% amino acid sequence homology to the isolated SARP of claim 1, 2, 3, 4, 5, 6, or 7.

13. A fragment of an isolated and purified SARP wherein said fragment selectively binds SA, ASA or 2,6-DHBA.

14. The fragment of claim 8 wherein said fragment comprises SEQ ID 1, 2, or 102.

15. The isolated and purified SARP of claim 1 wherein the protein comprises SEQ ID NO: 1.

16. An expression vector encoding the protein sequence of SARP.

17. An isolated and purified unglycosylated SARP protein.

18. A kit comprising an antibody that selectively binds SARP in its A state and an antibody that selectively binds SARP in its B state.

19. A drug delivery complex comprising SARP coupled to a therapeutic agent wherein the therapeutic agent is released from the complex when the SARP is in its state B and wherein the therapeutic agent is maintained in the complex when the SARP is in its state A.

20. A method for treating a metabolic condition in a subject comprising administering to the subject an agent that maintains SARP in a single state for a therapeutic period of time.

21. An isolated and purified salicylic acid receptor protein (SARP) comprising a polypeptide of ˜34.1 kDa, wherein the protein comprises at least one binding site for a ligand, the ligand selected from the group consisting of
(a) a salicylate-related compound,
(b) a nucleotide triphosphate that is selected from ATP and GTP.
(c) a nicotiamide adenine dinucleotide that is selected from NAD, and NADP.

22. An isolated SARP comprising a polypeptide of ˜34.1 kDa that is isolated from a biological sample by a process comprising the steps of:
(a) extracting the biological sample in a solvent to obtain a soluble extract free from insoluble material, wherein the solvent is selected from the group consisting of
(i) solution comprising at least 25% isopropanol by volume
(ii) solution comprising at least 50% isopropanol by volume
(iii) solution comprising at least 50% isopropanol by volume
(iv) solution comprising at least 95% isopropanol by volume
(v) solution comprising isopropanol: acetone: toluene at a ratio of 1:2:1
(vi) solution having a solvent polarity/polarizability (SPP) scale value of from ˜0.655 to 0.900; and
(b) resolving SARP from the soluble extract by a reversed-phase separation, wherein the protein comprises at least one binding site for a ligand, the ligand selected from the group consisting of
(i) a salicylate-related compound,
(ii) a nucleotide triphosphate that is selected from ATP and GTP.
(iii) a nicotiamide adenine dinucleotide that is selected from NAD, and NADP.

23. SARP of either claim 21 or claim 22 wherein the absence of a salicylate-related compound has an ESI-MS spectra with maxima at m/z 854, 900, 924, 1178, 1220, and where in the presence of a salicylate-related compound, a shift in the ESI-MS spectra comprise a global conformational change.

24. SARP of claim 23 wherein the conformational change occurs at a m/z 500 to 2000.

25. SARP of either claim 21 or claim 22 wherein in the absence of a salicylate-related compound the protein has no detectable absorbance spectrum, and wherein in the presence of a salicylate-related compound the protein comprises a detectable absorbance spectrum.

26. SARP of claim 25 wherein the absorbance spectrum occurs between 190 nm and 280 nm.

27. SARP of either claim 21 or claim 22, wherein the protein is capable of transferring at least one electron to an electron acceptor in the presence of a salicylate-related compound.

28. SARP of claim 27 wherein the protein is capable of transferring two electrons to an electron acceptor.

29. SARP of claim 28 wherein the electron acceptor is selected from the group consisting of MTT, NBT and Neo-NBT, and cytochrome c.

30. A protein according to either claim 21 or claim 22 that is isolated from a thermogenic plant that belongs to a family selected from the group consisting of Araceae, Nymphaeaceae, Cycadaceae, and Brassicaceae.

31. A protein according to claim 30 wherein the plant is Sauromatum guttatum.

32. The protein of claim 31, wherein digestion of the polypeptide with trypsin results in at least one peptide fragment that comprises the amino acid sequence set forth in SEQ ID NO:1 [FLPSEFGNDVDR].

33. A protein according to claim 30 wherein the plant is selected from the group consisting of Arum italicum, Amorphophallus konjac, Dracunculus vulgaris, Arabidopsis thaliana.

34. A protein according to claim 30 wherein the plant is Victoria cruziana.

35. A protein according to claim 30 wherein the plant is Encepalartos feros.

36. A protein according to claim 30 wherein the plant is Arabidopsis thaliana.

37. The protein of either claim 21 or claim 22 wherein the salicylate-related compound is selected from the group consisting of salicylic acid, acetylsalicylic acid, 2,6-DHBA, benzoic acid, 2,1,3-benzothiadiazole, and 3,4-dihydroxybenzoic acid.

38. A protein according to either claim 21 or claim 22 that forms a gel in water, the gel being capable of condensing and relaxing in a periodic manner.

39. A protein according to claim 38 that condenses and relaxes in the presence of a salicylate-related compound.

40. A protein according to either claim 21 or claim 22 that is capable of self-assembly into an oligomeric structures.

41. The protein of claim 40 wherein the oligomeric structure comprises a linear structure when self-assembly, and wherein the oligomeric structure comprises a micellar, rod, film, and dendrimer structures

42. A method of identifying an agent that alters a salicylate biological effect, comprising
(a) contacting an isolated SARP with a candidate agent under conditions and for a time sufficient to permit interaction between said SARP and said agent, wherein
(i) SARP comprises a polypeptide of ˜34.1 kDa having a ESI-MS spectra with maxima at m/z 854, 900, 924, 1178, 1220 in the absence of a salicylate-related compound and which comprises at least one shift in SARP in the presence of a salicylate-related compound, and
(ii) SARP comprises at least one binding site for a ligand that is selected from the group consisting of a salicylate-related compound and a nucleotide triphosphate which is selected from ATP and GTP; and NAD(P), NAD(P)H.
(b) comparing a level of binding of a ligand to SARP in the absence of the candidate agent to the level of binding of the ligand to the SARP in the presence of the agent, wherein an altered level of binding in the presence of the agent indicates the agent alters a salicylate biological effect.

43. A method of identifying an agent that alters a salicylate biological effect, comprising:
(a) contacting an isolated SARP with an electron acceptor molecule and a candidate agent under conditions and for a time sufficient to permit transfer of at least one electron from SARP to the electron acceptor, wherein
(i) SARP comprises a polypeptide of ˜34.1 kDa having a ESI-MS spectra with maxima at m/z 854, 900, 924, 1178, 1220 in the absence of a salicylate-related compound and which comprises at least one conformational change in the ESI-MS spectra in the presence of a salicylate-related compound, and
(ii) SARP comprises at least one binding site for a ligand that is selected from the group consisting of a salicylate-related compound and a nucleotide triphosphate which is selected from ATP and GTP; and NAD(P) and NAD(P)H.
(b) comparing a level of electron transfer from SARP to an electron acceptor in the absence of the candidate agent to the level of transfer in the presence of the agent, wherein an altered level of electron transfer in the presence of the agent indicates the agent alters a salicylate biological effect.

44. A method of identifying an agent that alters a salicylate biological effect, comprising:
(a) contacting an isolated SARP with a candidate agent under conditions and for a time sufficient to permit interaction between said SARP and said agent, wherein
(i) SARP comprises a polypeptide of ˜34.1 kDa having a ESI-MS spectra with maxima at m/z 854, 900, 924, 1178, 1220 in the in the absence of a salicylate-related compound and which comprises at least one conformational change in the ESI-MS spectra in the presence of a salicylate-related compound, and
(ii) SARP comprises at least one binding site for a ligand that is selected from the group consisting of a salicylate-related compound and a nucleotide triphosphate which is selected from ATP and GTP; and wherein altered absorbance of light by the protein in the presence of the agent relative to the absorbance of light by the protein in the absence of the agent indicates the agent alters a salicylate biological effect.

45. A method of identifying an agent that alters a salicylate biological effect, comprising:
(a) contacting an isolated SARP with a candidate agent under conditions and for a time sufficient to permit interaction between said SARP and said agent, wherein
(i) SARP comprises a polypeptide of ˜34.1 kDa having an absorbance spectrum which comprises no detectable UV absorbance in the absence of a salicylate-related compound and which comprises a detectable UV absorbance in the presence of a salicylate-related compound, and
(ii) SARP comprises at least one binding site for a ligand that is selected from the group consisting of a salicylate-related compound and a nucleotide triphosphate which is selected from ATP and GTP; and NAD(P), NAD(P)H.
(b) comparing, in the absence and presence of the candidate agent, an oligomeric state of SARP at one or a plurality of time points, wherein an altered oligomeric state of the protein in the presence of the agent relative to the oligomeric state of the protein in the absence of the agent indicates the agent alters a salicylate biological effect.

46. The method of any one of claims 41 -44 wherein SARP is isolated from a thermogenic plant.

47. The method of any one of claims 41 -44 wherein SARP is isolated from a mammal.

48. The method of claim 47 wherein the mammal is selected from the group consisting of a primate, a leporida (rabbit), a caviida (guinea pig), a rodent, a bovida and a suida (pig).

49. An antibody that specifically binds a SARP, wherein said SARP is selected from the group consisting of the protein of claim 21 and the protein of claim 22.

50. A protein complex for biological delivery of an agent, comprising a plurality of isolated SARP, wherein:
(i) SARP comprises a polypeptide of ˜34.1 kDa having an absorbance spectrum which comprises no detectable UV spectrum in the absence of a salicylate-related compound and which comprises a change in the UV spectrum in the presence of a salicylate-related compound,
(ii) SARP comprises at least one binding site for a ligand that is selected from the group consisting of a salicylate-related compound and a nucleotide triphosphate which is selected from ATP and GTP, and
(iii) the complex is present in an oligomeric state that is selected from the group consisting of a gel and an ordered aggregate.

51. A protein complex for biological delivery of an agent, comprising a gel which comprises a plurality of isolated SARP, wherein:
(i) the salicylic acid SARP protein comprises a polypeptide of ˜34.1 kDa having an absorbance spectrum which comprises no detectable isosbestic point in the absence of a salicylate-related compound and which comprises at least one isosbestic point in the presence of a salicylate-related compound, and
(ii) the salicylic acid SARP protein comprises at least one binding site for a ligand that is selected from the group consisting of a salicylate-related compound and a nucleotide triphosphate which is selected from ATP and GTP.

52. A method of identifying an agent that alters a salicylate biological effect, comprising:
(a) contacting an isolated SARP with a candidate agent under conditions and for a time sufficient to permit interaction between said SARP protein and said agent, wherein
(i) SARP comprises a plant reductase polypeptide selected from plant polypeptides comprising amino acid sequences set forth in the group consisting of SEQ ID NOS:3-101 said plant reductase polypeptide having an ESI-MS spectra with maxima at m/z 854, 900, 924, 1178, 1220 which comprises at least one change in the magnidute of protein ions in the presence of a salicylate-related compound, and
(ii) SARP comprises at least one binding site for a ligand that is selected from the group consisting of a salicylate-related compound and a nucleotide triphosphate which is selected from ATP and GTP; and NAD(P), NAD(P)H.
(b) comparing a level of binding of a ligand to SARP in the absence of the candidate agent to the level of binding of the ligand to SARP in the presence of the agent, wherein an altered level of binding in the presence of the agent indicates the agent alters a salicylate biological effect.

53. A method of identifying an agent that alters a salicylate biological effect, comprising:
(a) contacting an isolated SARP, an electron acceptor molecule and a candidate agent under conditions and for a time sufficient to permit transfer of at least one electron from SARP to the electron acceptor, wherein
(i) SARP comprises a plant reductase polypeptide selected from plant polypeptides comprising amino acid sequences set forth in the group consisting of SEQ ID NOS: 3-101, said plant reductase polypeptide having having an ESI-MS spectra with maxima at m/z 854, 900, 924, 1178, 1220 in the absence of a salicylate-related compound and which comprises a hnge in the magnidute of ion proteins in the presence of a salicylate-related compound, and
(ii) SARP comprises at least one binding site for a ligand that is selected from the group consisting of a salicylate-related compound and a nucleotide triphosphate which is selected from ATP and GTP; and NAD(P), NAD(P)H.
(b) comparing a level of electron transfer from SARP to the electron acceptor in the absence of the candidate agent to the level of transfer in the presence of the agent, wherein an altered level of electron transfer in the presence of the agent indicates the agent alters a salicylate biological effect.

54. A method of detecting an altered level of temperature in a plant, comprising detecting at two or more time points a level of SARP at a location in a biological sample comprising a cell derived from the plant, wherein said location is selected from a cell-associated location and a non-cell-associated location, and wherein an a change in conformation of SARP at the location at a later time point relative to the level of SARP at an earlier time point indicates an altered temperature level.

55. A method of detecting an altered level of resistance to a pathogen in a plant, comprising detecting at two or more time points a level of SARP at a location in a biological sample comprising a cell derived from the plant, wherein said location is selected from a cell-associated location and a non-cell-associated location, and wherein an increase in the level and conformation of SARP at the location at a later time point relative to the level of SARP at an earlier time point indicates an altered resistance level.

56. A method of altering a salicylate biological effect in a plant, comprising contacting the plant with a protein selected from the protein of claim 21, the protein of claim 2, and a plant reductase polypeptide that is selected from plant polypeptides comprising amino acid sequences set forth in the group consisting [SEQ ID NOS:3-101], wherein the salicylate biological effect comprises resistance to frost damage.
